(19) United States
(12) Patent Application Publication
(10) Pub. No.: US 2023/0263881 A1
(43) Pub. Date: Aug. 24, 2023

(54) Vaccine compositions for SARS-related coronaviruses and methods of use

(71) Applicants: Richard ASCIONE, Jersey City, NJ (US); Shengmei QI, Jersey City, NJ (US); David HODGE, Sedona, AZ (US)

(72) Inventors: Richard ASCIONE, Jersey City, NJ (US); Shengmei QI, Jersey City, NJ (US); David HODGE, Sedona, AZ (US)

(21) Appl. No.: 17/920,191

(22) PCT Filed: Apr. 20, 2021
(86) PCT No.: PCT/US21/28172

§ 371 Date: Oct. 28, 2021

Related U.S. Application Data
(60) Provisional application No. 63/012,847, filed on Apr. 20, 2020.

(51) Int. Cl. A61K 39/215; A61P 31/14

(52) U.S. Cl. CPC A61K 39/215; A61P 31/14; A61K 2039/55566

(57) ABSTRACT

The invention provides a pan-Severe Acute Respiratory Syndrome (SARS) vaccine compositions (i.e., vaccine compositions useful against multiple SARS viruses such as MERS, SARS-CoV-2, etc.), a vaccination regimen for immunization against such coronavirus diseases, and its use in medicine and in augmenting immune responses to various antigens present in such viruses and to methods of preparation of such compositions. In particular, the invention relates to polyvalent multi-targeting immunogenic compositions comprising SARS-coronaviral antigens or antigen preparations thereof from multiple strains associated with human pandemic outbreaks in combination with accessory delivery vehicle(s) and adjuvants.

### CROSS REFERENCE TO RELATED APPLICATIONS

The present application is a U.S. National Phase Application under 35 U.S.C. § 371 of International Patent Application No. PCT/US21/28172 filed Apr. 20, 2021, which claims priority to U.S. Provisional Patent Application No. 63/012,847, filed on Apr. 20, 2020, the entire contents of each of which are herein incorporated by reference.

### SEQUENCE LISTING

The instant application contains a Sequence Listing which has been submitted electronically in ASCII format and is hereby incorporated by reference in its entirety. Said ASCII copy, created on Apr. 19, 2021, is named 259205_000004_SL.txt and is 21,856 bytes in size.

### TECHNICAL FIELD

The present invention relates to a pan-Severe Acute Respiratory Syndrome (SARS) vaccine compositions (i.e., vaccine compositions useful against multiple SARS viruses such as MERS, SARS-CoV-2, etc.), a vaccination regimen for immunization against such coronavirus diseases, and its use in medicine and in augmenting immune responses to various antigens present in such viruses and to methods of preparation of such compositions. In particular, the invention relates to polyvalent multi-targeting immunogenic compositions comprising SARS-coronaviral antigens or antigen preparations thereof from multiple strains associated with human pandemic outbreaks in combination with accessory delivery vehicle(s) and adjuvants.

### BACKGROUND

Pandemic SARS-related coronaviruses (S-CoV) have caused worldwide epidemics periodically in past years, with infections ranging from mild respiratory illnesses to those causing major pulmonary complications resulting in severe morbidity and significant numbers of deaths, particularly in the elderly and very young individuals; especially those immuno-compromised as well as those with underlying chronic diseases. Modeling after flu vaccinations that play an important role, primarily in controlling and preventing major epidemic outbreaks, there is a need to formulate and use current technology to achieve vaccines that would prevent such periodic pandemics.

Currently, commercial methods consist of: (i) producing effective vaccines, in particular for respiratory RNA vaccines like influenza vaccines; and/or (ii) deployment of inactivated virus as vaccine or using the somewhat less effective live attenuated virus as vaccines. Vaccines for use in both methods are typically prepared on a large scale in embryonated chicken eggs, or, of late, in mammalian cell cultures. Given the present urgent need for the 2019 strain Coronavirus (SARS-CoV-2) vaccines, when many are now attempting vaccines for preventing the current epidemic from spreading further, this invention provides a method that does not require large scale coronavirus or use of eggs and cellular systems for production. Instead, it is possible to formulate and manufacture the invention's immunogenic vaccine compositions by simple chemistry and design them to be protective against many strains of coronaviruses that have been associated with human disease.

Emergence of worldwide S-CoV pandemics (e.g., SARS-1, MERS; SARS-CoV-2), even though somewhat frequent, are nevertheless severe and tend to be devastating in terms of mortality and morbidity especially in undeveloped countries where vaccines and antiviral resources are limited. Such pandemic outbreaks, are notoriously difficult to prepare for in advance by typical vaccine strategies, because of their unpredictable specific surface antigen sequences. Additionally, using current cell or egg-based technologies to produce commercial viral vaccines predicates at least a 6-8-month lead time being required for any pandemic vaccine, in which time the viral disease could likely afflict about a third of any population before becoming available for prophylaxis (K. A. McLean et. al., Vaccine 34 (45), 5410-5413 “The 2015 Global Production Capacity of Seasonal and Pandemic Influenza Vaccine” (2016)). This pandemic scenario would of necessity arise as a consequence of the limitations in current virus production methods requiring multi-millions or even billions of chicken eggs, as well as with the existing limited complex isolation-containment services required for maintaining sterile animal cell culture facilities for live and/or inactivated potentially lethal viruses needed for (any pandemic) future coronavirus vaccines. Thus, effective pandemic coronavirus vaccines are ones desirable for long lasting immunity against any emerging coronaviruses, that have recombined in zoonotic hosts and mutated to form highly lethal strains of other species of lethal (to humans) coronaviruses, like the current SARS-CoV-2 that has associated with it an overall lethality of about 4% or 20 fold higher than the current influenza viruses (S. Kalman et al., “COVID-19 (Novel Coronavirus 2019)—recent trends”, Eur Rev Med Pharmacol Sci. 2020; 24(4):2006-2011).

The vaccines needed for this type of prophylaxis must, in and of themselves, be safe and harmless to individuals handling and receiving them; use of the pathogen itself would be precluded for such vaccine preparations given its highly lethal nature. Frequently such preparations may also pose difficulties in yields of viruses, given the highly pathogenic nature of these viruses, which tend to inhibit growth in host embryos or cells, thereby compounding the difficulties for a ready and available supply of sufficient vaccines for worldwide distribution.

An alternative approach to afford immunologic protection against a variety of S-CoV viruses is to employ short immunogenic peptide regions shared by these pathogenic organisms, which would correspond to immunologically identifiable regions in the organism known as epitopes. Such peptide epitopes can be constructed synthetically and combined into a multivalent vaccine that could provoke immunologic antibody protection against collective S-CoV pathogens, including mutants or variants of the viruses, without the need for utilizing any actual pathogen components that would require strict safety/isolation procedures.

A major difficulty of small peptide epitope vaccines, however, are that they are notoriously poor immunogens and require larger carrier proteins to evoke substantial antibody and T-cell responses. Additionally, these peptide vaccines do not often provoke substantial enough immune responses or are of such low affinity and titers that they are ineffectual at binding to the native pathogen because of their inability to retain a recognizable spatial/stable conformation for substantial periods of time to evoke a proper immune response or even to have that immune response access and bind to the natural pathogenic epitope. Yet an advantage to working with such peptide-epitopes vaccines, is that they can be synthesized quickly and directed towards epitopes that are not normally frequently mutated or immunodominant; a problem that does not frequently occur when using the native whole pathogenic virus as a vaccine component.

### BRIEF SUMMARY OF THE INVENTION

As specified in the Background Section, there is a great need in the art to identify technologies for vaccine compositions designed to provide immunity against a variety of S-CoV viruses, including variants of existing viruses such as the SARS-CoV-2 virus, and use this understanding to develop novel vaccine compositions and methods of administering same. The present invention satisfies this and other needs. Embodiments of the present invention relate generally to vaccine compositions comprising peptide epitopes that are common to many S-CoV viruses, and more specifically to vaccine compositions comprising peptide epitopes that are not normally frequently mutated or immunodominant in combination with immunogenic components, such as for example and not limitation, immunogenic carriers, adjuvants, and short peptides covalently joined to the S-CoV peptides.

In a first aspect, the invention provides a composition comprising low amounts of S-CoV viral antigens that are associated with several SARS-related pandemic strains or would have the potential of associating with any future SARS-related pandemic viruses. In some specific embodiments the suitable antigens are short peptides or short peptide mimics of select ‘spike’ antigenic sequences and/or select nucleocapsid protein, as well as other viral regions all highly conserved in the known, sequenced (D. Wrapp, et al. “Cryo-EM structure of the 2019-nCoV spike in the prefusion conformation”. Science. 2020; 367(6483):1260-1263) pandemic coronaviruses; (SARS-2/CoV-2019; SARS-1, MERS as well as several known Bat coronaviruses) that are thought to be precursor mammalian zoonotic hosts. At least one or more of these peptide epitopes are not located in immunodominant surface regions of the coronavirus virions or in regions known to mutate frequently. Suitably these S-CoV antigenic epitopes are peptides typically less than 30 amino acid residues, which can be chemically synthesized by standard GMP solid phase synthesis methods known to the art.

In a second aspect of this invention, the suitable S-CoV antigens are peptides or peptide mimics of select antigenic sequences conserved in the angiotensin converting enzyme (ACE)-exodomain host receptor binding regions of the known, sequenced pathogenic coronaviruses (SARS-2/CoV-2019/SARS-CoV-2; SARS-1, MERS as well as several known Bat and beta-coronaviruses). At least one or more of these S-CoV short peptide epitopes are within the pathogenic S-CoV host's receptor binding domains (RBD) and/or are in the spike protein domain regions adjacent and these epitopes and/or are located in the S-CoV nucleocapsid structure surrounding the RNA genome and not generally located in immunodominant surface regions known to mutate frequently. Suitably, these S-CoV antigenic epitopes are non-toxic peptides, chemically synthesized by standard GMP solid phase synthesis methods known to the art.

In a third aspect, the invention provides intrinsic non-viral specific spacer peptides operably linked to the viral epitopes in order to permit the proper peptide antigen orientation to the immune system following conjugation of peptide antigens-spacer complex onto the specific protein carrier that constitutes the immunogen and enables the sufficient and sustained persistence of said immunogen to effect useful titers of specific epitope targeting antibody of S-CoV protein regions. The conjoiner spacer peptide comprises four or more amino acid residues, e.g., four or more proline residues, further comprising a cysteine residue preferably at the C or N-terminus depending on the orientation of the antigen epitope desired for conjugation to carrier protein(s). Preferably, there are two hydroxy amino acids (e.g., S or T such as SS, TT, TS, or ST) that are conjugated to the opposite terminus from the cysteine residue. Exemplary conjoiner spacer peptides can have D-enantiomeric forms of one or more of the amino acids. Preferred conjoiner spacer peptides include SS, TT, TS, or ST conjugated to ppppC (SEQ ID NO: 38; wherein the lowercase amino acids are D-enantiomers, for use at the C-terminal end of the epitopes discussed herein) and Cpppp (SEQ ID NO: 39) conjugated to SS, TT, TS, or ST, wherein the lowercase amino acids are D-enantiomers, for use at the N-terminal end of the epitopes discussed herein). The linkage of the peptide spacer to the carrier should be stable under physiologic conditions and not interfere with the desired epitope in the antigenic peptide. Moreover, the conjoined spacer peptides afford resistance to proteolytic degradation that extends the circulatory life of these therapeutic peptides enhancing immunity and allowing persistence for antigen presenting cell (APC) presentation. Conjugation to the protein carrier in the invention is by any conventional conjugation method known in the art, and using peptide covalent conjugation chemistry methods known to the art.

In a fourth aspect, the invention provides combinations of one or more peptide antigenic epitopes. In some embodiments, the vaccine compositions comprise two or more peptide antigenic epitopes, three or more peptide antigenic epitopes, four or more peptide antigenic epitopes, or five or more peptide antigenic epitopes, optionally coupled to immunogenic carriers. In some embodiments, the vaccine compositions comprise at least five or more peptide antigenic epitope immunogens, optionally coupled to immunogenic carriers (comprising multiple RBD and/or comprising epitopes of spike protein domain regions adjacent to locating S-CoV RBD epitopes and/or epitopes located in one or more of the nucleocapsid protein, Membrane matrix protein, Envelope protein, and/or Replicase protein) to form the polyvalent anti-pandemic immunogenic composition. Mixing of these polyvalent components is based on antibody titers against epitope antigens, determined by standard immunological methods known to the art and compositions comprising the epitopes can include further components, e.g., carriers, adjuvants, and/or excipients. Nonlimiting exemplary combinations of epitopes described herein include at least 3-5 peptides, optionally coupled to comprising amino acid sequences selected from the group consisting of SEQ ID NOs:1-20 and 26-31 and/or amino acid sequences and/or mimetic sequences selected from the group consisting of SEQ ID NOs: 21-25 and 34-36 and/or SEQ ID: 32, 33, and 37, optionally coupled to immunogenic carriers. Additional nonlimiting exemplary combinations include at least 3 amino acid sequences selected from the group consisting of SEQ ID NOs: 10, 11, 12, 14, 15, 26, 28, 30 and 31, optionally coupled to immunogenic carriers, and at least 3 amino acid sequences selected from the group consisting of SEQ ID NOs: 10, 14, 15, 28, 30 and 31, optionally coupled to immunogenic carriers.

In a fifth aspect, the invention provides a combination of at least five or more peptide antigenic epitope immunogens, optionally coupled to immunogenic carriers (comprising multiple SARS-CoV-2 Nucleocapsid residues and/or comprise epitopes of nucleocapsid protein domain regions adjacent to the Viral RNA components of the S-CoV and/or epitopes located in one or more of the spike protein, RBD, Membrane matrix protein, Envelope protein, and/or Replicase protein) to form the polyvalent anti-pandemic immunogenic composition. Mixing of these polyvalent components is based on antibody titers against epitope antigens, determined by standard immunological methods known to the art.

In a sixth aspect, the invention provides a combination of the at least five or more peptide antigenic epitope immunogens, optionally coupled to immunogenic carriers (comprising multiple SARS-CoV-2 virion residues and/or comprise epitopes of antibody accessible domain virion protein regions of the S-CoV) to form the polyvalent anti-pandemic immunogenic composition. Mixing of these polyvalent components is based on antibody titers against epitope antigens, determined by standard immunological methods known to the art.

In a seventh aspect, the invention provides the combination of any of the polyvalent immunogenic compositions disclosed herein in a water-in-oil vehicle that forms a stable emulsion for extended periods of time, with or without added specific adjuvant(s), using standard emulsification techniques known to the art. The emulsion can be in the form of a nanoemulsion, in which 50% or more of the nanoparticles have a diameter of 250 nanometers or less. Aluminum or calcium phosphate-based adjuvants are commonly used in the art, however non-metallic salt-based adjuvants can also be used.

In an eighth aspect, the invention provides for the inoculation of the immunogenic composition by different routes, known to the art using suitable dosing regimens determined by taking into account the factors well known to the art, including age, weight, sex and medical status of the patient as well as the route chosen for immunogenic administration. Exemplary administration routes include subcutaneous, intramuscular, mucosal (e.g., intranasal or sublingual). The specific aspect of this pandemic immunogenic composition requires no reformulation of polyvalent components from year to year, but the periodic boosting of the vaccines at intervals to be determined by serological titers against antigenic epitopes by standard immunological methods known in the art.

In a ninth aspect, the invention provides for a method that would by using the formulated immunogenic composition(s) prevent, delay, reduce, inhibit or otherwise restrict the coronaviral infections in clinical applications for such subjects so exposed. More specifically the invention comprises administration to subjects effective dosages of immunogenic composition(s) that contain such conserved non-mutated spike domain epitopes, spacers, conjugated to immunogenic carriers thereof, comprising a vaccine to elicit multiple, protective antibody titers that would afford sustained prophylactic defense against pandemic S-CoV beta-coronaviral infection.

### BRIEF DESCRIPTION OF THE FIGURES

The accompanying Figures, which are incorporated in and constitute a part of this specification, illustrate several aspects described below.

FIG. 1 shows the viral titer in rabbits who received 30 μg of SEQ ID NO: 7 over a 72-day time course. All titers were performed by standard ELISA assays using serial dilutions of rabbit antisera on cognate (SEQ ID NO: 7 for FIG. 1, respective peptides for other SEQ ID as indicated) epitope peptides with an alanine spacer coupled to bovine serum albumin carrier proteins affixed to commercial microwell-plates, and binding detected using commercial secondary murine-anti-rabbit antibody conjugated to horseradish peroxidase (HRP) for colorimetric (chromogenic) detection that are standard for substrates in ELISA applications for HRP.

FIG. 2 shows the viral titer in rabbits who received 45 μg of SEQ ID NO: 11 over a 72-day time course.

FIG. 3 shows the viral titer in rabbits who received 30 μg of SEQ ID NO: 12 over a 72-day time course.

FIG. 4 shows the viral titer in rabbits who received 30 μg of SEQ ID NO: 14 over a 72-day time course.

FIG. 5 shows the viral titer in rabbits who received 30 μg of SEQ ID NO: 15 over a 72-day time course.

FIG. 6 shows the viral titer in rabbits who received 45 μg of SEQ ID NO: 20 over a 72-day time course.

FIG. 7 shows the viral titer in rabbits who received 45 μg of SEQ ID NO: 23 over a 72-day time course.

FIG. 8 shows the viral titer in rabbits who received 45 μg of SEQ ID NO: 24 over a 72-day time course.

FIG. 9 shows the viral titer in rabbits who received 45 μg of SEQ ID NO: 25 over a 72-day time course.

FIG. 10 shows the viral titer in rabbits who received 30 μg of SEQ ID NO: 8 over a 72-day time course.

### DETAILED DESCRIPTION OF THE INVENTION

As specified in the Background Section, there is a great need in the art to identify technologies for vaccine compositions designed to provide immunity against a variety of S-CoV viruses, including variants of existing viruses such as the SARS-CoV-2 virus, and use this understanding to develop novel vaccine compositions and methods of administering same. The present invention satisfies this and other needs. Embodiments of the present invention relate generally to vaccine compositions comprising peptide epitopes that are common to many S-CoV viruses, and more specifically to vaccine compositions comprising peptide epitopes that are not normally frequently mutated or immunodominant in combination with immunogenic components, such as for example and not limitation, immunogenic carriers, adjuvants, and short peptides covalently joined to the S-CoV peptides.

To facilitate an understanding of the principles and features of the various embodiments of the invention, various illustrative embodiments are explained below. Although exemplary embodiments of the invention are explained in detail, it is to be understood that other embodiments are contemplated. Accordingly, it is not intended that the invention is limited in its scope to the details of construction and arrangement of components set forth in the following description or examples. The invention is capable of other embodiments and of being practiced or carried out in various ways. Also, in describing the exemplary embodiments, specific terminology will be resorted to for the sake of clarity.

It must also be noted that, as used in the specification and the appended claims, the singular forms “a,” “an” and “the” include plural references unless the context clearly dictates otherwise. For example, reference to a component is intended also to include composition of a plurality of components. References to a composition containing “a” constituent is intended to include other constituents in addition to the one named. In other words, the terms “a,” “an,” and “the” do not denote a limitation of quantity, but rather denote the presence of “at least one” of the referenced item.

As used herein, the term “and/or” may mean “and,” it may mean “or,” it may mean “exclusive-or,” it may mean “one,” it may mean “some, but not all,” it may mean “neither,” and/or it may mean “both.” The term “or” is intended to mean an inclusive “or.”

Also, in describing the exemplary embodiments, terminology will be resorted to for the sake of clarity. It is intended that each term contemplates its broadest meaning as understood by those skilled in the art and includes all technical equivalents which operate in a similar manner to accomplish a similar purpose. It is to be understood that embodiments of the disclosed technology may be practiced without these specific details. In other instances, well-known methods, structures, and techniques have not been shown in detail in order not to obscure an understanding of this description. References to “one embodiment,” “an embodiment,” “example embodiment,” “some embodiments,” “certain embodiments,” “various embodiments,” etc., indicate that the embodiment(s) of the disclosed technology so described may include a particular feature, structure, or characteristic, but not every embodiment necessarily includes the particular feature, structure, or characteristic. Further, repeated use of the phrase “in one embodiment” does not necessarily refer to the same embodiment, although it may.

As used herein, the term “about” should be construed to refer to both of the numbers specified as the endpoint (s) of any range. Any reference to a range should be considered as providing support for any subset within that range. Ranges may be expressed herein as from “about” or “approximately” or “substantially” one particular value and/or to “about” or “approximately” or “substantially” another particular value. When such a range is expressed, other exemplary embodiments include from the one particular value and/or to the other particular value. Further, the term “about” means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to ±5%, and more preferably still up to ±1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Similarly, as used herein, “substantially free” of something, or “substantially pure”, and like characterizations, can include both being “at least substantially free” of something, or “at least substantially pure”, and being “completely free” of something, or “completely pure”.

By “comprising” or “containing” or “including” is meant that at least the named compound, element, particle, or method step is present in the composition or article or method, but does not exclude the presence of other compounds, materials, particles, method steps, even if the other such compounds, material, particles, method steps have the same function as what is named.

Throughout this description, various components may be identified having specific values or parameters, however, these items are provided as exemplary embodiments. Indeed, the exemplary embodiments do not limit the various aspects and concepts of the present invention as many comparable parameters, sizes, ranges, and/or values may be implemented. The terms “first,” “second,” and the like, “primary,” “secondary,” and the like, do not denote any order, quantity, or importance, but rather are used to distinguish one element from another.

It is noted that terms like “specifically,” “preferably,” “typically,” “generally,” and “often” are not utilized herein to limit the scope of the claimed invention or to imply that certain features are critical, essential, or even important to the structure or function of the claimed invention. Rather, these terms are merely intended to highlight alternative or additional features that may or may not be utilized in a particular embodiment of the present invention. It is also noted that terms like “substantially” and “about” are utilized herein to represent the inherent degree of uncertainty that may be attributed to any quantitative comparison, value, measurement, or other representation.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “50 mm” is intended to mean “about 50 mm.”

It is also to be understood that the mention of one or more method steps does not preclude the presence of additional method steps or intervening method steps between those steps expressly identified. Similarly, it is also to be understood that the mention of one or more components in a composition does not preclude the presence of additional components than those expressly identified.

The materials described hereinafter as making up the various elements of the present invention are intended to be illustrative and not restrictive. Many suitable materials that would perform the same or a similar function as the materials described herein are intended to be embraced within the scope of the invention. Such other materials not described herein can include, but are not limited to, materials that are developed after the time of the development of the invention, for example. Any dimensions listed in the various drawings are for illustrative purposes only and are not intended to be limiting. Other dimensions and proportions are contemplated and intended to be included within the scope of the invention.

As used herein, the term “subject” or “patient” refers to mammals and includes, without limitation, human and veterinary animals. In a preferred embodiment, the subject is human.

The terms “treat” or “treatment” of a state, disorder or condition include: (1) preventing or delaying the appearance of at least one clinical or sub-clinical symptom of the state, disorder or condition developing in a subject that may be afflicted with or predisposed to the state, disorder or condition but does not yet experience or display clinical or subclinical symptoms of the state, disorder or condition; or (2) inhibiting the state, disorder or condition, i.e., arresting, reducing or delaying the development of the disease or a relapse thereof (in case of maintenance treatment) or at least one clinical or sub-clinical symptom thereof; or (3) relieving the disease, i.e., causing regression of the state, disorder or condition or at least one of its clinical or sub-clinical symptoms. The benefit to a subject to be treated is either statistically significant or at least perceptible to the patient or to the physician.

As used herein the term “therapeutically effective” applied to dose or amount refers to that quantity of a compound or pharmaceutical composition that when administered to a subject for treating (e.g., preventing or ameliorating) a state, disorder or condition, is sufficient to effect such treatment. The “therapeutically effective amount” will vary depending on the compound or bacteria or analogues administered as well as the disease and its severity and the age, weight, physical condition and responsiveness of the mammal to be treated.

The phrase “pharmaceutically acceptable”, as used in connection with compositions of the invention, refers to molecular entities and other ingredients of such compositions that are physiologically tolerable and do not typically produce untoward reactions when administered to a mammal (e.g., a human) Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans.

The terms “pharmaceutical carrier” or “pharmaceutically acceptable carrier” refer to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Alternatively, the pharmaceutical carrier can be a solid dosage form carrier, including but not limited to one or more of a binder (for compressed pills), a glidant, an encapsulating agent, a flavorant, and a colorant. Suitable pharmaceutical carriers are described in “Remington's Pharmaceutical Sciences” by E. W. Martin.

In the context of the field of medicine, the term “prevent” encompasses any activity which reduces the burden of mortality or morbidity from disease. Prevention can occur at primary, secondary and tertiary prevention levels. While primary prevention avoids the development of a disease, secondary and tertiary levels of prevention encompass activities aimed at preventing the progression of a disease and the emergence of symptoms as well as reducing the negative impact of an already established disease by restoring function and reducing disease-related complications.

In the context of the present invention, the term “vaccine” (also referred to as an immunogenic composition, vaccine composition, polyvalent composition, polyvalent immunogen, or vaccine formulation) refers to a substance that induces anti-S-CoV immunity or suppresses S-CoV virus upon inoculation into a subject.

### DESCRIPTION OF THE INVENTION

The present invention deals with the development of effective protective vaccines and vaccine-like immunogens that can target the beta-coronaviruses that have emerged in the past two decades and include the current SARS2-CoV/COVID-19/SARS-CoV-2 as well as the recent SARS1 and MERS agents and is based upon selecting peptides that result in production of antibodies that bind to non-mutated, conserved epitopes within the spike protein region that is responsible for binding to the viruses host ACE2 receptors for entry.

The invention is based upon the selection of short peptides that are in highly conserved S-CoV RBD and/or are in the spike protein domain regions adjacent in protein specific regions of most all known pathogenic S-CoV type viruses. Normally such short peptide regions (“epitopes”) are poorly immunogenic and thus unable to stimulate any immune system to mount any significant, protective antibody responses against such short peptide-epitope targets. However, the inventors have found that it is possible to modify such epitope sequences by chemical synthesis coupling to a unique short peptide spacer and chemically conjugating these components to a carrier protein that renders these peptide epitopes immunostimulatory.

### Compositions of the Invention

In some embodiments, the vaccine compositions comprise a peptide antigenic epitope, two or more peptide antigenic epitopes, three or more peptide antigenic epitopes, four or more peptide antigenic epitopes, or five or more peptide antigenic epitopes, optionally coupled to immunogenic carriers. In some embodiments, the vaccine compositions comprise at least five or more peptide antigenic epitope immunogens, optionally coupled to immunogenic carriers (comprising multiple RBD and/or comprising epitopes of spike protein domain regions adjacent to locating S-CoV RBD epitopes and/or epitopes located in one or more of the nucleocapsid protein, Membrane matrix protein, Envelope protein, and/or Replicase protein) to form the polyvalent anti-pandemic immunogenic composition. In some embodiments, the peptides of the composition are 6 to 50 amino acids in length, preferably from 6 to 30 amino acids in length.

In some embodiments, the peptides of the composition comprise spacer peptides comprising two hydroxy amino acids (e.g., S or T) coupled to a ppppC (SEQ ID NO: 38) or Cpppp (SEQ ID NO: 39) spacer, which is used to couple the viral peptide to an immunogenic carrier. The orientation of the spacer peptide is selected based on the location of the fusion to the immunogenic carrier.

In an embodiment, the spike protein amino acid sequence is about 15 amino acids and is located amino-terminal to probable receptor binding residues. In another embodiment, the spike protein amino acid sequence is about 20 amino acids and is located proximal the probable receptor binding domain (RBD) residues. In another embodiment, the spike protein amino acid sequence is about 19 amino acids and is located amino-terminal to fusion residues. In another embodiment, the spike protein amino acid sequence is about 20 amino acids and is located amino-terminal to fusion residues. In another embodiment, the spike protein amino acid sequence is about 20 amino acids and is located carboxy-terminal to fusion residues. In another embodiment, the spike protein amino acid sequence is about 17 amino acids and is located in the amino-terminal N-Terminal Domain (NTD) region. In an embodiment, the spike protein amino acid sequence is about 16 amino acids and is located in the carboxy-terminal region in NTD. In an embodiment, the spike protein amino acid sequence is about 18 amino acids and is located amino-terminal in heptad repeat (HR) residue region. In an embodiment, the spike protein amino acid sequence is about 20 amino acids and is located in HR carboxy-terminal region.

In another embodiment, the nucleocapsid (NC) protein amino acid sequence is about 23 amino acids and is located in the NC amino-terminal region. In an embodiment, the nucleocapsid (NC) protein amino acid sequence is about 18 amino acids and is located in NC carboxy-terminal region. In an embodiment, the nucleocapsid (NC) protein amino acid sequence is about 22 amino acids and is located in NC carboxy-terminal region.

In another embodiment, the Membrane matrix (M) protein amino acid sequence is about 24 amino acids and is located in the amino-terminus of the M region.

In another embodiment, the Envelope protein amino acid sequence is about 18 amino acids and is located in the amino terminal region.

In another embodiment, the Replicase protein amino acid sequence is about 19 amino acids and is located in the carboxy-terminal region of the polyprotein S-CoV RNA polymerase.

In an embodiment, vaccine compositions according to the present invention comprise one or more peptides comprising SEQ ID NOs: 1-37, optionally coupled to an immunogenic carrier, preferably two or more peptides comprising SEQ ID NOs: 1-37, optionally coupled to an immunogenic carrier, more preferably three or more peptides comprising SEQ ID NOs: 1-37, optionally coupled to an immunogenic carrier, more preferably four or more peptides comprising SEQ ID NOs: 1-37, optionally coupled to an immunogenic carrier, and most preferably five or more comprising SEQ ID NOs: 1-37, optionally coupled to an immunogenic carrier. A vaccine composition according to the present invention can comprise additional ingredients, such as pharmaceutically acceptable carriers, adjuvants, and/or excipients.

In another embodiment, the vaccine compositions comprise at least five peptides comprising SEQ ID NOs: 1-37, optionally coupled to an immunogenic carrier.

Nonlimiting exemplary combinations of epitopes described herein include at least 3-5 peptides comprising amino acid sequences selected from the group consisting of SEQ ID NOs:1-20 and 26-31 and/or amino acid sequences and/or mimetic sequences selected from the group consisting of SEQ ID NOs: 21-25 and 34-36 and/or SEQ ID: 32, 33, and 37, optionally coupled to immunogenic carriers. Additional nonlimiting exemplary combinations include at least 3 amino acid sequences selected from the group consisting of SEQ ID NOs: 10, 11, 12, 14, 15, 26, 28, 30 and 31, optionally coupled to immunogenic carriers, and at least 3 amino acid sequences selected from the group consisting of SEQ ID NOs: 10, 14, 15, 28, 30 and 31, optionally coupled to immunogenic carriers.

In the following amino acid sequences, capital letters represent L-isomer amino acids and lowercase letters represent D-isomer amino acids, where F* represents para-nitro-phenylalanine—a Y mimetic, and where M* is L-Norleucine (nLeu) the L-amino acid mimic for L-Methionine.
1. An immunogenic composition comprising a S-CoV ‘spike’ protein mimetic peptide RGVYYPD-TTppppC (SEQ ID NO: 1), optionally coupled to an immunogenic carrier.
2. An immunogenic composition comprising a S-CoV ‘spike’ protein mimetic peptide RGWIFG-STppppC (SEQ ID NO: 2), optionally coupled to an immunogenic carrier.
3. An immunogenic composition comprising a ‘spike’ protein S-CoV mimetic peptide LREFVFKN-SSppppC (SEQ ID NO: 3), optionally coupled to an immunogenic carrier.
4. An immunogenic composition comprising a ‘spike’ protein S-CoV mimetic peptide RFPNITN-SSppppC (SEQ ID NO: 4), optionally coupled to an immunogenic carrier.
5. An immunogenic composition comprising a ‘spike’ protein S-CoV mimetic peptide: PFGEVFNA-TTppppC (SEQ ID NO: 5) coupled to an immunogenic carrier.
6. An immunogenic composition comprising a ‘spike’ protein S-CoV mimetic peptide: VADYSVLF*N-TSppppC (SEQ ID NO: 6), optionally coupled to an immunogenic carrier.
7. An immunogenic composition comprising a ‘spike’ protein S-CoV mimetic peptide: CppppTS-SFSTFK (SEQ ID NO: 7), optionally coupled to an immunogenic carrier.
8. An immunogenic composition comprising a ‘spike’ protein S-CoV mimetic peptide: CppppTT-NVYADSFV (SEQ ID NO: 8), optionally coupled to an immunogenic carrier.
9. An immunogenic composition comprising a ‘spike’ protein S-CoV mimetic peptide: CppppSS-DEVRQIAPGQTG (SEQ ID NO: 9), optionally coupled to an immunogenic carrier.
10. An immunogenic composition comprising a ‘spike’ protein S-CoV mimetic peptide: IADF*NYKLPDD-TTppppC (SEQ ID NO: 10), optionally coupled to an immunogenic carrier.
11. An immunogenic composition comprising a ‘spike’ protein S-CoV mimetic peptide: RPFERDIS-TTppppC (SEQ ID NO: 11), optionally coupled to an immunogenic carrier.
12. An immunogenic composition comprising a ‘spike’ protein S-CoV mimetic peptide: QPF*RVVVLSFELI-SSppppC (SEQ ID NO: 12), optionally coupled to an immunogenic carrier.
13. An immunogenic composition comprising a ‘spike’ protein S-CoV mimetic peptide: GGVSVITPGTN-TSppppC (SEQ ID NO: 13), optionally coupled to an immunogenic carrier.
14. An immunogenic composition comprising a ‘spike’ protein S-CoV mimetic peptide: DIPIGAGISASYH-TSppppC (SEQ ID NO: 14), optionally coupled to an immunogenic carrier.
15. An immunogenic composition comprising a ‘spike’ protein S-CoV mimetic peptide: AYTM*SLGA-SSppppC (SEQ ID NO.: 15), optionally coupled to an immunogenic carrier.
16. An immunogenic composition comprising a ‘spike’ protein S-CoV mimetic peptide: NNSIAIPTNFTI-SSppppC (SEQ ID NO.: 16), optionally coupled to an immunogenic carrier.
17. An immunogenic composition comprising a ‘spike’ protein S-CoV mimetic peptide: CppppSS-NLLLQF*GSF (SEQ ID NO.: 17), optionally coupled to an immunogenic carrier.
18. An immunogenic composition comprising a ‘spike’ protein S-CoV mimetic peptide: GGFNFSQILPDP-SSppppC (SEQ ID NO.: 18), optionally coupled to an immunogenic carrier.
19. An immunogenic composition comprising a ‘spike’ protein S-CoV mimetic peptide: CppppSS-RSFIED (SEQ ID NO.: 19), optionally coupled to an immunogenic carrier.
20. An immunogenic composition comprising a ‘spike’ protein S-CoV mimetic peptide: CppppTT-STALGKLQD (SEQ ID NO.: 20), optionally coupled to an immunogenic carrier.
21. An immunogenic composition comprising a ‘Nucleocapsid’ protein S-CoV mimetic peptide: GVPINTNS-TTppppC (SEQ ID NO.:21), optionally coupled to an immunogenic carrier.
22. An immunogenic composition comprising a ‘Nucleocapsid’ protein S-CoV mimetic peptide: CppppSS-PRWYFYF* (SEQ ID NO.:22), optionally coupled to an immunogenic carrier.
23. An immunogenic composition comprising a ‘Nucleocapsid’ protein S-CoV mimetic peptide: GTRNPANN-SSppppC (SEQ ID NO.:23), optionally coupled to an immunogenic carrier.
24. An immunogenic composition comprising a ‘Nucleocapsid’ protein S-CoV mimetic peptide: CppppSS-SRSSSRSR (SEQ ID NO.:24), optionally coupled to an immunogenic carrier.
25. An immunogenic composition comprising a ‘Nucleocapsid’ protein S-CoV mimetic peptide: IDAYKTFP-TTppppC (SEQ ID NO.:25), optionally coupled to an immunogenic carrier.
26. An immunogenic composition comprising a ‘Spike’ protein S-CoV mimetic peptide: SKRSFVENLLFNK-TTppppC (SEQ ID NO.:26), optionally coupled to an immunogenic carrier.
27. An immunogenic composition comprising a ‘Spike’ protein S-CoV mimetic peptide: TLADVGFM*KQYDD-STppppC (SEQ ID NO.:26), optionally coupled to an immunogenic carrier.
28. An immunogenic composition comprising a ‘Spike’ protein S-CoV mimetic peptide: CppppST-AGAAAYYVGF* (SEQ ID NO.:28), optionally coupled to an immunogenic carrier.
29. An immunogenic composition comprising a ‘Spike’ protein S-CoV mimetic peptide: CppppSS-DPLSETKST (SEQ ID NO.:29), optionally coupled to an immunogenic carrier.
30. An immunogenic composition comprising a ‘Spike’ protein S-CoV mimetic peptide: QKLIANQFNSA-SSppppC (SEQ ID NO.:30), optionally coupled to an immunogenic carrier.
31. An immunogenic composition comprising a ‘Spike’ protein S-CoV mimetic peptide: HTSPDVDLGDISG-TTppppC (SEQ ID NO.:31), optionally coupled to an immunogenic carrier.
32. An immunogenic composition comprising a ‘Membrane (matrix)’ protein S-CoV mimetic peptide: NGTITVEELKKLLEQWN-TTppppC (SEQ ID NO.:32), optionally coupled to an immunogenic carrier.
33. An immunogenic composition comprising an ‘Envelope’ protein S-CoV mimetic peptide: SEETGTLIVNS-TTppppC (SEQ ID NO.:33), optionally coupled to an immunogenic carrier.
34. An immunogenic composition comprising a ‘Nucleocapsid’ protein S-CoV mimetic peptide: CppppTT-PNNTASWFTALTQHGK (SEQ ID NO.:34), optionally coupled to an immunogenic carrier.
35. An immunogenic composition comprising a ‘Nucleocapsid’ protein S-CoV mimetic peptide: KHINAYKTFPP-TTppppC (SEQ ID NO.:35), optionally coupled to an immunogenic carrier.
36. An immunogenic composition comprising a ‘Nucleocapsid’ protein S-CoV mimetic peptide: QRQKKQQTVTLLPAA-STppppC (SEQ ID NO.:36), optionally coupled to an immunogenic carrier.
37. An immunogenic composition comprising a ‘REPLICASE’ protein S-CoV mimetic peptide: NLNRGM*VLGSLA-STppppC (SEQ ID NO.:37), optionally coupled to an immunogenic carrier.
38. The immunogenic composition of SEQ ID NOs: 1-37, wherein the immunogenic carrier is selected from the group consisting of: diphtheria toxoid (DT), tetanus toxoid (TT), pertussin toxoid (PT), BCG-proteins, Pure Protein Derivative (PPD), Measles, Mumps, Rubella, Varicella viral proteins, recombinant-TT, recombinant-HepB, HPV-VLP, and as carrier protein, any FDA approved commercial pharmaceutical vaccine GMP produced protein(s).
39. The immunogenic composition of SEQ ID NOs: 1-37, wherein the immunogenic carrier is a recombinant expressed immunogenic polypeptide larger than 3000 Daltons (˜≥300 amino acids).
40. A pharmaceutical composition comprising a therapeutically effective amount of the immunogenic composition of SEQ ID NOs: 1-37 and a pharmaceutically acceptable carrier to effect disease protective titers in the majority of hosts vaccinated either intramuscularly, subcutaneously, or mucosally (including intranasally or sublingually).
41. The pharmaceutical composition of SEQ ID NOs: 1-37 and item 38-40, wherein the pharmaceutically acceptable carrier comprises an emulsion of an aqueous phase and an oily phase, wherein the oily phase is an emulsified oily vehicle comprising squalene, squalane, Polysorbate 80, Sorbitan monooleate, Polysorbate 40, vitamin E, Vitamin D3, ascorbyl palmitate, and combinations thereof.
42. The pharmaceutical composition of claim SEQ ID NOs: 1-37 and item 38-41, wherein the pharmaceutically acceptable carrier comprises suitably acceptable vehicle such as an emulsion of an aqueous phase and an oily phase, wherein the oily phase is an oily vehicle comprising squalene, squalane, Polysorbate 80, Sorbitan monooleate, Polysorbate 40, and combinations thereof, and wherein either the oily or aqueous phase contains adjuvants comprising, but not limited to: nano-particulate aluminum or calcium phosphate, diguanylate cyclic monophosphates, guanosine, (cyclic-di-GMP) or other cyclic-di mononucleotides that activate STING immunological pathways, Praziquantel, Imiquimod, Nor-MDP, Ergamisol, Cimetidine, uric acid, cyclic diguanylate, threonyl-N-acetyl-muramyl-L-alanyl-D-isoglutamine, Isoprinosine, mannan, trehalose dimycolate, QS-21 and alpha-galactosylceramide (α-GalCer) or alphaglucosylceramide (α-GluCer).

The invented compositions can elicit disease ameliorating and/or protective adaptive antibodies and/or T-cell responses able to recognize non-mutated, highly conserved epitopes in the spike protein regions and/or the nucleocapsid region that may be cryptic in the intact virion and not readily recognized by conventional vaccination procedures using inactivated or attenuated coronaviruses, due to the presence of the more immunodominant and highly mutable surface spike epitopes.

The invented compositions can elicit disease ameliorating and/or protective adaptive antibodies and/or T-cell responses that are able to recognize non-mutated, highly conserved non-mutated epitope regions that have not been elicited from conventional whole viruses, inactivated or attenuated, or by recombinant coronaviral proteins, but which is widely present in all beta (β) type coronaviruses, such as the SARS-1, MERS, SARS-2_CoV viruses.

The invention provides immunogens that can elicit high antibody titers, which can offer broad protection against infection, particularly across many of the varied types and strains of known β-corona viruses that infect humans. The invention can afford immunological protection against pandemic viruses that have not altered the conserved cryptic sites in the past 10+ years. The invention also is readily and easily modifiable by alteration of easily prepared peptide epitopes and can be used to elicit other antibodies for any number of emerging pathogens that threaten human populations.

The invention describes usage of non-pathogenic, non-viral immunogen components that are chemically synthesized by standard GMP chemistries known in the art. These components do not require the use of pathogenic organisms, or infected embryonated eggs, or even require cells and complex, sterile media and containment facilities that widely used viral vaccine productions currently utilize. The methods required for these non-cellular preparations can result in a purer vaccine preparation with no contaminating viruses that can be introduced using animal cell cultures. Also, the heterogeneity of targeted epitopes, particularly those in the coronaviral proteins that one would experience using live chicken embryonated viral products, is precluded by this invention. In the absence of live viruses, as such, there is no need to employ denaturation agents or organic solvents to kill and inactivate or to extract virus material for vaccines and thus the invention avoids denaturing antigenic residues, retaining immunogenic epitopes as well as precluding other safety concerns that could arise from the use of such chemicals and that would likely remain as residuals. Individuals with egg allergies and chemical sensitivities (e.g., denaturing, formaldehyde containing compounds) would not have to be used and thus the absence of such materials in these synthetic peptide vaccines that contain no live cellular or viral components thus avoids hypersensitivity issues that arise with commonly used viral vaccines in current commercial production.

The invention provides a broadly protective pan-coronavirus vaccine that could be protective for all pandemic viral strains thus far known that have emerged over the last two decades; a vaccine that is easily manufactured from readily obtainable peptide materials without the need for live cell or virus materials and can yield a highly purified product that could be considerably more cost efficient to produce due to the absence of containment facilities that commercial vaccines produced in avian eggs or in live animal cells currently require.

The inventors have discovered that modified amino acids in epitopic regions (referred to herein as mimic-epitopes or “mimitopes”) that can be used to enhance the recognition of “foreign” antigens and therefore activate intrinsic and adaptive immune systems accordingly to engender greater anti-viral immune responses. More specifically, it is recognized that the human natural innate immune system in its processing by neutrophils, monocytes and macrophages (collectively known as antigen presentation cells (APCs)) are able to modify the microbial antigens by peroxidation, halogenation and nitrosylation reactions. Such innate immune chemical modifications are known by the art to enhance and augment the antigenicity and subsequent immunogenicity of the microbial antigens. Accordingly, the present invention includes and encompasses the employment of one or more halogenated or nitrogenated amino acid residues, such as para-nitro-phenylalanine or para-chloro-phenylalanine to augment the immunogenicity of the immunogen. For example, para-L-nitrophenylalanine (nPhe/F*) is listed in certain of our inventive mimic epitope residues (mimitopes) (as in SEQ ID NOs: 6, 10, 12, 17, 22 and 28); similarly, L-Norleucine (nLeu/M*) is listed in certain of our inventive mimic epitope residues (mimitopes) (as in SEQ ID NOs: 15, 27, and 37) detailed herein.

The inventors have discovered that certain smaller peptide mimetics (less than 30 amino acid residues) would be able to enhance their recognition by APCs, by spatially orienting themselves especially with more resistant residues to proteolysis; likely allowing a longer period for antigen presentations. Therefore, the invented compositions include peptides comprising a series of D-amino acid residues of proline as spacers placed adjacent to the mimotopes in all 37 of the listed sequences herein.

According to one aspect of the invention, therefore, the improved mimitope immunogens can generate multitargeting polyclonal antibodies against viral receptor binding domain regions and their N-terminal and/or C-terminal spike protein species, respectively.

A non-limiting example of such an immunogen is one that comprises:
(i) a peptide of the amino acid residues: an immunogen composition comprising a S-CoV ‘spike’ protein mimetic peptide: Ser-Asn-Ile-Ile-Arg-Gly-Trp-Ile-Phe-Gly-Thr-Thr-pro-pro-pro-pro-Cys (SEQ ID NO.: 1); coupled to (ii) an immunogenic carrier.

Accordingly, this embodiment incorporates a spike protein component N-terminal region-mimitope (residues 98-107 of Spike Protein YP_009724390) plus a 7 amino-acid spacer (e.g., SS, TT, TS, or ST coupled to ppppC (SEQ ID NO: 38) or Cpppp (SEQ ID NO: 39) to constitute an immunomimic that is a 17 amino-acid peptide. Similarly, this embodiment incorporates a spike protein component C-terminal region-mimitope (residues 856-869 of Spike Protein YP_009724390) plus a 7 amino-acid spacer to constitute an immunomimic that is a 21 amino-acid peptide.

In the present invention, the immunogenic carrier can be any suitable, high molecular-weight carrier, typically a protein or large (i.e., generally greater than 6000 kD) molecule of sufficient molecular complexity that can elicit an immune response towards a haptene or peptide sequence that is covalently linked to it. The category of suitable immunogenic carriers is exemplified by but not limited to toxoidal proteins like diphtheria toxoid (DT), tetanus toxoid (TT), pertussin toxoid (PT), or recombinant toxoids like PrimeBio Inc., novel, non-toxic but immunogenic recombinant Tetanus (drTeNT) protein. Among these, tetanus toxoid is a preferred immunogenic carrier. This category also encompasses particulate carriers such as the nanoparticulate calcium phosphate (nCAP) described by Qing He et al., Clin. Diag. Lab Immunology 7:899-903 (2000).

A suitably acceptable vehicle denotes a medically safe, non-toxic substance that will convey an immunogen without diminishing its immunogenic effect. A suitable vehicle therefore, can be a liquid emulsion, as further described below, or it can be a stable particulate substance, e.g., as a pharmaceutically safe lyophilized powder or pharmaceutically acceptable hydrocolloidal gel or recombinant, synthesized, non-infectious virus like particles (VLP) that are now FDA approved in commercial vaccines. See FIELDS VIROLOGY, 6^{th} ed. Vol. I, D. M. Knipe & P. Howley (eds.), Lippincott Williams & Wilkins (2013).

In an embodiment, a preferred form of pharmaceutically acceptable vehicle is an emulsion of an aqueous phase, containing the polypeptide immunogen, and an oily phase. The oily phase comprises at least one biodegradable oil, immiscible with the aqueous phase, that is non-toxic in the dosage range of intended administration. The oil can be natural or synthetic, and there are many such oils available, which are generally recognized as safe and meet international regulatory acceptance for therapeutic vaccine use. Illustrative of such suitable oils are squalene, squalane, Sorbitan monooleate, Polysorbate 40, and Polysorbate 80. A preferred oily phase comprises four or five of these component oils in the oily phase.

The emulsions are mixed for a sufficient amount of time to generate nanoparticles. The preferable size of a majority (i.e., more than about 50%) of the nanoparticles in the emulsion is 250 nanometers or less. For example, squalene/squalane-based emulsions can be mixed in an ice water bath at kept at (0-4° C.) low temperatures, using an IKA overhead stirrer suitable for 8,000 rpm mixing and/or Microfluidics M-110P Microfluidizer™ mixer or equivalent, for a time period suitable for generating nano-sized emulsification particles.

In addition, the oily phase may contain one or more separate emulsifiers, such as alpha-tocopherol, aluminum monostearate or an adjuvant-active saccharide oleate or saccharide stearate ester.

In accordance with another aspect of the invention, either or both of the oily or aqueous phase of an emulsion as described above contains at least one adjuvant that is distinct from the immunogenic carrier component of the polypeptide immunogen. There is a wide range of known adjuvants, any one or more which may be considered for use in this invention.

Non-limiting examples of such known adjuvants are: cyclic-di-purine mononucleotides, cyclic diguanylate, Imiquimod, cyclic diadenylate, Isoprinosine, trehalose dimycolate, QS-21, alpha-galactosylceramide (C-GalCer), and alpha-glucosylceramide (C-GluCer). For this adjuvant role, moreover, the present invention comprehends the use of a material that, if not typically deemed an adjuvant per se, is immunostimulatory nevertheless. Exemplary of these materials are Ergamisol, Cimetidine, Praziquantel, uric acid, mannan and derivatives of mannan, and vitamins A, D3 and vitamin E.

In accordance with a further aspect of the invention, an improved immunogen generates polyclonal antibodies against the preferred consensus amino terminal epitope sequences of the beta-coronavirus spike protein, listed as YP_009724390. Illustrative of these immunogens are ones that comprises a peptide of the preferred sequence: Arg-Pro-Phe-Glu-Arg-Asp-Ile-Ser-Thr-Thr-pro-pro-pro-pro-Cys (SEQ ID NO: 11) coupled to an immunogenic carrier, as described above. Again, the non-toxic, more immunogenic (drTEnT®) tetanus recombinant protein is the preferred immunogenic carrier for this preferred embodiment.

A further embodiment of the invention is another preferred immunomimic that comprises a peptide of the sequence: Gln-Pro-nPhe-Arg-Val-Val-Val-Leu-Ser-Phe-Glu-Leu-Ile-Ser-Ser-pro-pro-pro-pro-Cys (SEQ ID NO. 12) coupled to an immunogenic carrier, as described above. Again, the non-toxic, more immunogenic (drTEnT®) recombinant Tetanus protein is the preferred immunogenic carrier in this embodiment.

As an additional embodiment, a spike protein peptide of the preferred sequence Asp-Ile-Pro-Ile-Gly-Ala-Gly-Ile-Ser-Ala-Tyr-His-Thr-Ser-pro-pro-pro-pro-Cys (SEQ ID NO: 14), coupled to an immunogenic carrier, as described above. The non-toxic, more immunogenic (drTEnT®) tetanus recombinant protein is the preferred immunogenic carrier for this preferred embodiment.

Still another preferred embodiment of the invention is an immunomimic comprising a spike peptide of the sequence: Ala-Tyr-Tr-nLeu-Ser-Leu-Gly-Ala-Ser-Ser-pro-pro-pro-pro-Cys (SEQ ID NO. 15) coupled to an immunogenic carrier, as described above. Again, the non-toxic, more immunogenic (drTEnT®) tetanus recombinant protein is the preferred immunogenic carrier for this preferred embodiment.

Still another preferred embodiment of the invention is an immunomimic comprising a spike peptide of the sequence: Cys-pro-pro-pro-pro-Thr-Thr-Ser-Thr-Ala-Leu-Gly-Lys-Leu-Gln-Asp (SEQ ID NO. 20) coupled to an immunogenic carrier, as described above. Again, the non-toxic, more immunogenic (drTEnT®) tetanus recombinant protein is the preferred immunogenic carrier for this preferred embodiment.

Another embodiment of the invention is a preferred immunomimic that comprises a S-CoV nucleocapsid peptide of the sequence: Gly-Thr-Arg-Asn-Pro-Ala-Asn-Asn-Ser-Ser-Ser-pro-pro-pro-pro-Cys (SEQ ID NO.: 23) coupled to a preferred immunogenic carrier, as described above. Accordingly, this mimic represents an improved immunogen able to generate polyclonal antibodies against the preferred consensus amino terminal epitope sequences of the beta-coronavirus Nucleocapsid protein, listed as QHD43423.2 in the NCBI database.

An embodiment of the invention is a preferred immunomimic that comprises a S-CoV nucleocapsid peptide of the sequence: Cys-pro-pro-pro-pro-Ser-Ser-Ser-Arg-Ser-Ser-Ser-Arg-Ser-Arg (SEQ ID NO.: 24) coupled to a preferred immunogenic carrier, as described above. Accordingly, this mimic represents an improved immunogen able to generate polyclonal antibodies against the preferred consensus amino terminal epitope sequences of the beta-coronavirus Nucleocapsid protein.

An embodiment is also a preferred immunomimic consisting of the S-CoV Nucleocapsid protein epitope sequences: Ile-Asp-Ala-Tyr-Lys-Thr-Phe-Pro-Thr-Thr-pro-pro-pro-pro-Cys (SEQ ID NO.: 25) containing spacers that contain one or more D-isomer prolyl amino acids, a feature can be important for proper positional presentation of adjacent immunogen peptides onto the carrier proteins.

An embodiment is also a preferred immunomimic consisting of the S-CoV Nucleocapsid protein epitope sequences: Gln-Arg-Gln-Lys-Lys-Gln-Gln-Thr-Val-Thr-Leu-Leu-Pro-Ala-Ala-Ser-Thr-pro-pro-pro-pro-Cys (SEQ ID NO.: 36) containing spacers that contain one or more D-isomer prolyl amino acids, a feature that can be important for proper positional presentation of adjacent immunogen peptides onto the carrier proteins.

An embodiment of the invention is a preferred immunomimic that comprises a S-CoV Membrane matrix peptide of the sequence: Asn-Gly-Thr-Ile-Thr-Val-Glu-Glu-Leu-Lys-Lys-Leu-Leu-Glu-Gln-Trp-Asn-Thr-Thr-pro-pro-pro-pro-Cys (SEQ ID NO.: 32) containing spacers that contain one or more D-isomer prolyl amino acids, a feature that can be important for proper positional presentation of adjacent immunogen peptides onto the carrier proteins.

An embodiment of the invention is a preferred immunomimic that comprises a S-CoV Envelope peptide of the sequence: Ser-Glu-Glu-Thr-Gly-Thr-Leu-Ile-Val-Asn-Ser-Thr-Thr-pro-pro-pro-pro-Cys (Seq ID NO: 33.) containing spacers that contain one or more D-isomer prolyl amino acids, a feature that can be important for proper positional presentation of adjacent immunogen peptides onto the carrier proteins.

Another embodiment of the invention is a preferred immunomimic that comprises a S-CoV Replicase peptide of the sequence: Asn-Leu-Asn-Arg-Gly-nLeu-Val-Leu-Gly-Ser-Leu-Ala-Ser-Thr-pro-pro-pro-pro-Cys (Seq ID NO: 37) containing spacers that contain one or more D-isomer prolyl acids, a feature that can be important for proper positional presentation of adjacent immunogen peptides onto the carrier proteins.

In the peptides described herein, the D-amino acid isomers can enable appropriate configuration for APC presentations, as well as enhance the persistence of the immunogen for APC presentation, yielding higher titers of antibody.

In conventional antibody technology, the induction of effective antibody responses by immunization with immune active conjugated carrier complexes, typically requires two or more administrations of immunogen, and it takes several weeks or months for the antibody titers to rise to the desired levels. By contrast, the improved immunogenic compositions of the present invention may induce effective levels of antibody shortly after the administration of initial course of immunogen. Levels of antibody thus elicited may stay elevated for several months and readily elevate to higher levels upon subsequent boosting by a single injection of an immunogenic composition according to the invention.

In accordance with one aspect of the invention, the novel anti-pan coronaviral immunogenic composition could generate polyclonal antibodies against conserved beta-coronaviral spike protein species. Illustrative of such an immunogenic composition is one that comprises (i) a selection of at least two, three or more mimitopes represented by SEQ ID NOs: 1-20, and 26-31 and listed above covalently coupled to (ii) an immunogenic carrier, suspended in a nano-emulsion and (iii) formulated using an aqueous and oily phase as described above, respectively.

Similarly, another aspect of the invention is a novel anti-pan coronaviral immunogenic composition could generate polyclonal antibodies against a selection of conserved beta-coronaviral nucleocapsid protein species. Illustrative of such an immunogenic composition is one that comprises (i) a selection of at least two or more mimitopes represented by SEQ ID NOs: 21-25, and 34-36 listed above covalently coupled to (ii) an immunogenic carrier, suspended in a nano-emulsion and (iii) formulated using an aqueous and oily phase as described above, respectively.

Also, another aspect of the invention is a novel anti-pan coronaviral immunogenic composition that would be desired would generate polyclonal antibodies against conserved beta-coronaviral membrane matrix protein species. Illustrative of such an immunogenic composition is one that comprises (i) a selection of at least one or more mimitopes represented by SEQ ID NO: 32 listed along with others above covalently coupled peptides to (ii) an immunogenic carrier, suspended in a nano-emulsion and (iii) formulated using an aqueous and oily phase as described above, respectively.

In another aspect of the invention, the novel anti-pan coronaviral immunogenic composition could generate polyclonal antibodies against conserved beta-coronaviral Envelope protein species. Illustrative of such an immunogenic composition is one that comprises (i) a selection of at least one or more mimitopes represented by SEQ ID NO: 33 listed along with above covalently coupled peptides to (ii) an immunogenic carrier, suspended in a nano-emulsion and (iii) formulated using an aqueous and oily phase as described above, respectively

Finally, in another aspect of the invention, the novel anti-pan coronaviral immunogenic composition could generate polyclonal antibodies against all conserved beta-coronaviral Replicase protein species. Illustrative of such an immunogenic composition is one that comprises (i) a selection of at least one or more mimitopes represented by SEQ ID NO: 37 listed along with above covalently coupled peptides to (ii) an immunogenic carrier, suspended in a nano-emulsion and (iii) formulated using an aqueous and oily phase as described above, respectively.

In the present invention, a preferred anti-pan coronaviral immunogenic composition would be to generate polyclonal antibodies against the beta-coronaviral spike protein species using an immunogen that comprises (i) a selection of at least three or more mimitopes selected from the group consisting of SEQ ID NOs: 10, 11, 12, 14, 15, 26, 28, 30 and 31 listed above covalently coupled to (ii) an immunogenic carrier, such as the preferred recombinant carriers of drTeNT or HPV-VLP described above and suspended in the nano-emulsion and (iii) formulated using an aqueous and oily phase as described above, respectively. In some embodiments, the mimitopes are selected from the group consisting of SEQ ID NOs: 10, 14, 15, 28, 30 and 31.

In the present invention, a preferred anti-pan coronaviral immunogenic composition could generate polyclonal antibodies against the beta-coronaviral nucleocapsid protein species using an immunogen that comprises (i) a selection of at least two or more mimitopes represented by SEQ ID NOs: 25, 34, 35 and 36 listed above covalently coupled to (ii) an immunogenic carrier, such as the preferred recombinant carriers of drTeNT or HPV-VLP described above and suspended in the nano-emulsion and (iii) formulated using an aqueous and oily phase as described above, respectively.

In the present invention, a preferred anti-pan coronaviral immunogenic composition could generate polyclonal antibodies against the beta-coronaviral conserved represented by a multiple selection of preferred spike and nucleocapsid sequences listed above along with SEQ ID NOs: 32 and 33 and covalently coupled to (ii) an immunogenic carrier, such as the preferred recombinant carriers of drTeNT or HPV-VLP described above and suspended in the nano-emulsion and (iii) formulated using an aqueous and oily phase as described above, respectively.

In any of the vaccine compositions disclosed herein, the immunogenic peptide(s) is/are present in a therapeutically effective amount. It is contemplated that the vaccine compositions are administered in two doses, e.g., a first priming dose and a second boosting dose. The amount of the immunogenic peptide(s) present in the two doses can be the same or different. The initial dosage can comprise about 25 to about 500 micrograms (μg) peptides (e.g., 25, 50 or 100 μg) followed in two to four weeks by a secondary dose of the same or double dosage as defined in clinical trial phase-1 dosage escalation studies.

### Exemplary Method of Making the Disclosed Immunogenic Compositions

In accordance with a preferred aspect of the invention, the peptides disclosed herein can be conjugated to amino groups present on the tetanus toxoid (TT) immunogenic carrier. The linkage was via the terminal peptide cysteine residue, utilizing heterobifunctional linking agents containing typically a succinimidyl ester at one end and maleimide at the other end of the linking agent. To accomplish the linkage between either of the Mimic Peptides listed above and the carrier, the cysteine of the peptide was first reduced. The dry peptide was dissolved in 0.1M sodium phosphate buffer, pH 7-9, with a 5-50 molar excess of dithiothreitol. The peptide was lyophilized and stored under vacuum until used. Typically, the carrier protein is activated by treatment with the hetero-bifunctional linking agent epsilon-maleimidocaproic acid N-hydroxysuccinimide ester (EMCS), in proportions sufficient to achieve activation of approximately 25 free amino groups per 105 molecular weight of carrier.

Preparation of Purified Tetanus Toxoid: Typically, if TT is used it was purified by ultrafiltration. Final concentration of recovered purified TT was expected to be 5-40 mg/ml. The purity was determined by chromatography (SEC HPLC), protein concentration (Bradford protein assay or Lowry assay), and free amino-groups (by ninhydrin). Peptides were obtained commercially (Biosyn Corp, USA), and reduced peptide with known purity and content was used for conjugation. Peptides were reduced with tris (2-carboxyethyl)-phosphine-HCl (TCEP), and the mixture was used in the conjugation. Ellman's assay can be used to determine free sulfhydryl groups.

Conjugation of Peptide-TT. After calculating the quantity of peptide to react with the maleimido-TT, the peptide was added to the M-TT solution. The peptide-TT conjugate was purified by ultrafiltration filtered. The conjugates of the peptides to be linked to carrier via EMCS and are to be separated from other components of the mixture by low pressure chromatography at 4° C. over a G50 Sephadex column equilibrated with 0.1-0.5M ammonium bicarbonate. In each case the conjugate was eluted in the column Void volume and was lyophilized and stored, desiccated, at 4-0° C. until use.

The conjugate may be characterized as to immunomimic peptide content by a number of methods known to those skilled in the art including weight gain, amino acid analysis, etc. Conjugates of peptides to carrier proteins produced by these methods are determined by amino acid analysis to have 10-30 moles of peptide per 104-106 MW of carrier and all are considered suitable as immunogens for immunization of test animals.

### Methods of Administering the Compositions

In a preferred embodiment, the vaccine compositions of the invention are administered in two doses, e.g., an initial dose followed by a second booster dose. For example, the second dose of the vaccine composition can be administered at least 2 weeks after the first dose, preferably is administered 3 weeks after the first dose, and more preferably is administered 4 weeks after the first dose. The amount of the disclosed peptides can be the same in the first dose and the second dose, or the amounts can be different. For example, the initial dosage can comprise about 25 to about 500 micrograms (μg) peptides (e.g., 25, 50 or 100 μg) followed in two to four weeks by a secondary dose of the same or double dosage as defined in clinical trial phase-1 dosage escalation studies.

The vaccine compositions can be administered by any desired route, e.g., subcutaneous, intramuscular, or mucosal (e.g., intranasal or sublingual) administration. The vaccine compositions can be formulated to be used in such administration methods.

The present invention is described further by reference to the following examples, which are illustrative only and not limiting of the invention.

### EXAMPLES

### Example 1: Immunogenicity of the Peptides Disclosed Herein

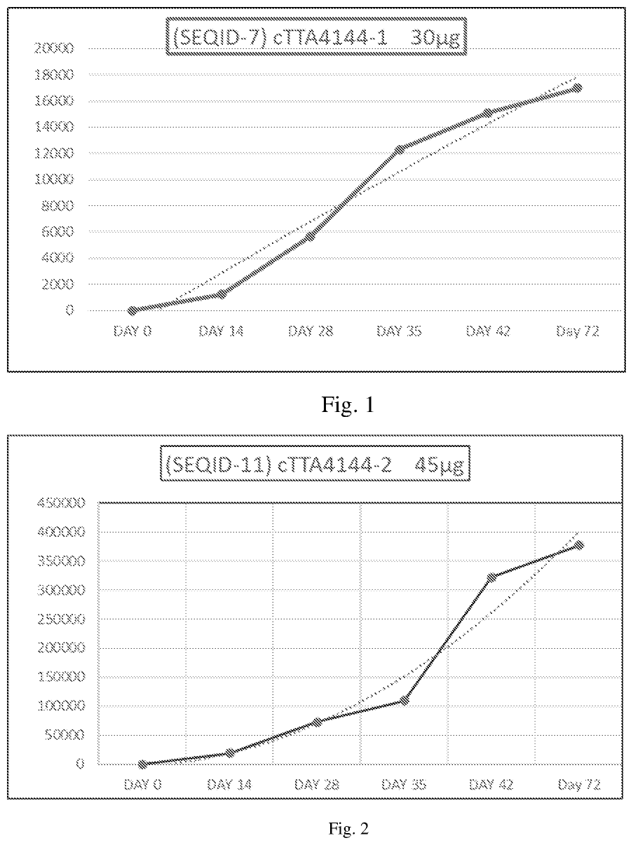

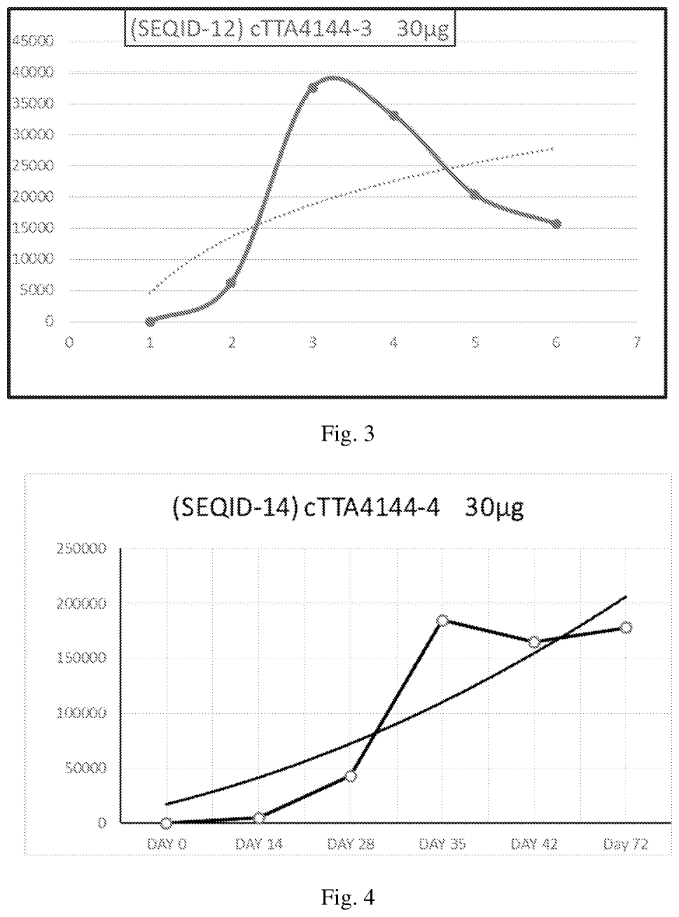

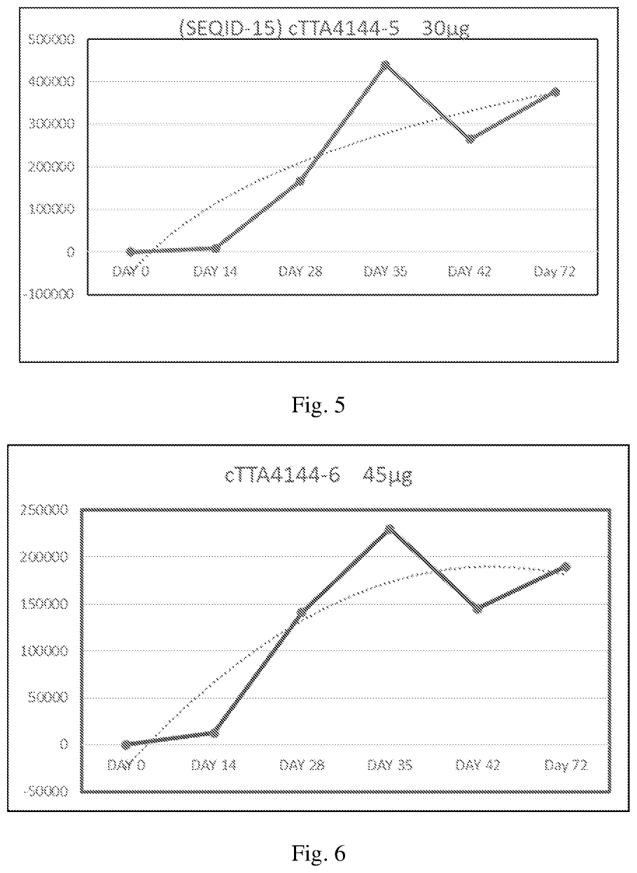

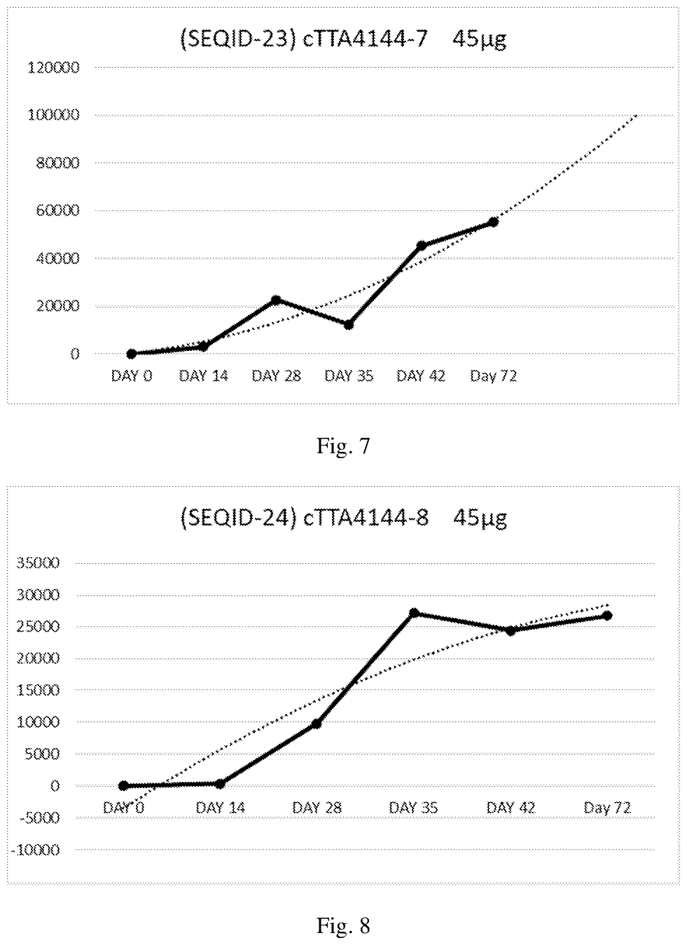

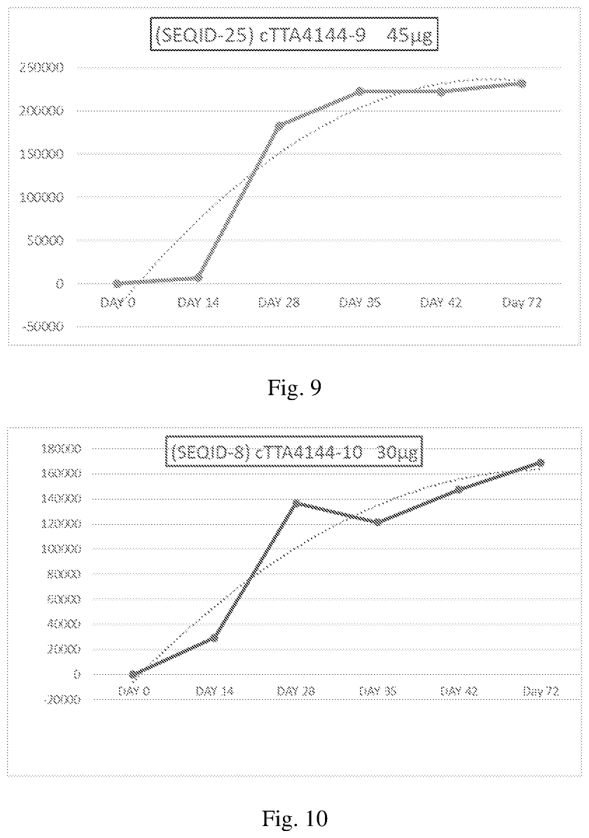

Japanese White rabbits were given intramuscularly the peptide compositions as described herein. Table 1 and FIGS. 1-10 show the viral titers elicited in the rabbits over a 72-day time course.

Thirty or 45 micrograms (μg) of each peptide in a water in oil emulsion (prepared as described) was used for the intramuscular immunization of each group of (three) Japanese White rabbits as indicated on day 0. Two weeks after the initial injection, each group of rabbits were given a second and third injection in amounts specified, at a 2-week interval (on days 14 and 28). No other booster injections were given. Measurement of antibody titers followed administration. Twenty-five to fifty ml of blood were collected from the rabbits each time at Days 35, 42 and 72 after the initial injection.

TABLE 1

| | DAY 0 | DAY 14 | DAY 28 | DAY 35 | DAY 42 | Day 72 |
|---|---|---|---|---|---|---|
| Group 1 cTTA4144-1 | | | | | | |
| (SEQID-7) cTTA4144-1 30 μg | 0 | 1,222 | 5,653 | 12,288 | 15,079 | 16,990 |
| Group 2 cTTA4144-2 | | | | | | |
| (SEQID-11) cTTA4144-2 45 μg | 0 | 19,155 | 72,911 | 109,145 | 321,704 | 377,088 |
| Group 3 cTTA4144-3 | | | | | | |
| (SEQID-12) cTTA4144-3 30 μg | 0 | 6,184 | 37,508 | 33,073 | 20,396 | 15,660 |
| Group 4 cTTA4144-4 | | | | | | |
| (SEQID-14) cTTA4144-4 30 μg | 0 | 4,467 | 42,743 | 184,686 | 164,632 | 177,895 |
| Group 5 cTTA4144-5 | | | | | | |
| (SEQID-15) cTTA4144-5 30 μg | 0 | 8,922 | 167,038 | 438,999 | 264,754 | 376,430 |
| Group 6 cTTA4144-6 | | | | | | |
| (SEQID-20) cTTA4144-6 45 μg | 0 | 12,456 | 140,936 | 230,124 | 144,805 | 189,772 |
| Group 7 cTTA4144-7 | | | | | | |
| (SEQID-23) cTTA4144-7 45 μg | 0 | 3,061 | 22,654 | 12,266 | 45,271 | 55,244 |
| Group 8 cTTA4144-8 | | | | | | |
| (SEQID-24) cTTA4144-8 45 μg | 0 | 346 | 9,832 | 27,186 | 24,431 | 26,778 |
| Group 9 cTTA4144-9 | | | | | | |
| (SEQID-25) cTTA4144-9 45 μg | 0 | 7,016 | 182,701 | 222,734 | 222,019 | 231,990 |
| Group 10 cTTA4144-10 | | | | | | |
| (SEQID-8) cTTA4144-10 30 μg | 0 | 29,346 | 136,734 | 121,245 | 147,514 | 168,955 |

### Example 2: Administration of the Vaccine Compositions

A vaccine composition as described herein can be administered in two doses. Preferably, the first dose is administered, and the second dose is administered at least two weeks after the first dose, at least three weeks after the first dose, or at least four weeks after the first dose. The amount of the disclosed peptides can be the same in the first dose and the second dose, or the amounts can be different. For example, the initial dosage can comprise about 25 to about 500 micrograms (μg) peptides (e.g., 25, 50 or 100 μg) followed in two to four weeks by a secondary dose of the same or double dosage as defined in clinical trial phase-1 dosage escalation studies.

The vaccine compositions can be administered by any suitable method, e.g., subcutaneously, intramuscularly, or mucosally (e.g., intranasal or sublingual).

### SEQUENCE LISTING

Peptides were prepared by standard solid-state synthesis commercial methods. Each peptide was characterized as to amino acid content and purity. Peptides with the amino acid sequences listed below were thus synthesized. In these sequences, as in others of the present description, an amino acid beginning in a capital letter is an L-isomer amino acid, while one in a lower-case letter is a D-isomer.

SEQ ID NO: 1. Arg-Gly-Val-Tyr-Tyr-Pro-Asp-Thr-Thr-pro-pro-pro-pro-Cys (14 aa). Represents a SARS2-CoV spike protein peptide mimitope partly homologous to NCBI-listed YP_009724390 residues 34-40.

SEQ ID NO: 2. Arg-Gly-Trp-Ile-Phe-Gly-Ser-Thr-pro-pro-pro-pro-Cys (13 aa). Represents a SARS2-CoV spike protein peptide mimitope partly homologous to NCBI-listed YP_009724390 residues 102-107.

Seq ID NO: 3. Leu-Arg-Glu-Phe-Val-Phe-Lys-Asn-Ser-Ser-pro-pro-pro-pro-Cys (15 aa). Represents a SARS2-CoV spike protein peptide mimitope partly homologous to NCBI-listed YP_009724390 residues 184-196.

Seq ID NO: 4. Arg-Phe-Pro-Asn-Ile-Thr-Asn-Ser-Ser-pro-pro-pro-pro-Cys (14 aa). Represents a SARS2-CoV spike protein peptide mimitope partly homologous to NCBI-listed YP_009724390 residues 328-335.

Seq ID NO: 5. Pro-Phe-Gly-Glu-Val-Phe-Asn-Ala-Thr-Thr-pro-pro-pro-pro-Cys (15 aa). Represents a SARS2-CoV spike protein peptide mimitope partly homologous to NCBI-listed YP_009724390 residues 337-344.

Seq ID NO: 6. Val-Ala-Asp-Tyr-Ser-Val-nPhe-Asn-Thr-Ser-pro-pro-pro-pro-Cys (16 aa). Represents a SARS2-CoV spike protein peptide mimitope partly homologous to NCBI-listed YP_009724390 residues 362-370.

Seq ID NO: 7. Cys-pro-pro-pro-pro-Thr-Ser-Ser-Phe-Ser-Thr-Phe-Lys (13 aa). Represents a SARS2-CoV spike protein peptide mimitope partly homologous to NCBI-listed YP_009724390 residues 373-378.

Seq ID NO: 8. Cys-pro-pro-pro-pro-Thr-Thr-Asn-Val-Tyr-Ala-Asp-Ser-Phe-Val (15 aa). Represents a SARS2-CoV spike protein peptide mimitope partly homologous to NCBI-listed YP_009724390 residues 394-401.

Seq ID NO: 9. Cys-pro-pro-pro-pro-Ser-Ser-Asp-Glu-Val-Arg-Gln-Ile-Ala-Pro-Gly-Gln-Thr-Gly (19 aa). Represents a SARS2-CoV spike protein peptide mimitope partly homologous to NCBI-listed YP_009724390 residues 405-416.

Seq ID NO: 10. Ile-Ala-Asp-nPhe-Asn-Tyr-Lys-Leu-Pro-Asp-Asp-Thr-Thr-pro-pro-pro-pro-Cys (18 aa). Represents a SARS2-CoV spike protein peptide mimitope partly homologous to NCBI-listed YP_009724390 residues 418-428.

Seq ID NO: 11. Lys-Pro-Phe-Glu-Arg-Asp-Ile-Ser-Thr-Thr-pro-pro-pro-pro-Cys (15 aa). Represents a SARS2-CoV spike protein peptide mimitope partly homologous to NCBI-listed YP_009724390 residues 462-469.

Seq ID NO: 12. Gln-Pro-nPhe-Arg-Val-Val-Val-Leu-Ser-Phe-Glu-Leu-Ile-Ser-Ser-pro-pro-pro-pro-Cys (20 aa). Represents a SARS2-CoV spike protein peptide mimitope partly homologous to NCBI-listed YP_009724390 residues 506-518.

Seq ID NO: 13. Gly-Gly-Val-Ser-Val-Ile-Thr-Pro-Gly-Thr-Asn-Thr-Ser-pro-pro-pro-pro-Cys (18aa). Represents a SARS2-CoV spike protein peptide mimitope partly homologous to NCBI-listed YP_009724390 residues 593-603.

Seq ID NO: 14. Asn-Ile-Pro-Ile-Gly-Ala-Gly-Ile-Ser-Ala-Tyr-His-Thr-Ser-pro-pro-pro-pro-Cys (19aa). Represents a SARS2-CoV spike protein peptide mimitope partly homologous to NCBI-listed YP_009724390 residues 663-674.

Seq ID NO: 15. Ala-Tyr-Thr-nLeu-Ser-Leu-Gly-Ala-Ser-Ser-pro-pro-pro-pro-Cys (18aa). Represents a SARS2-CoV spike protein peptide mimitope partly homologous to NCBI-listed YP_009724390 residues 694-700.

Seq ID NO: 16. Asn-Asn-Ser-Ile-Ala-Ile-Pro-Thr-Asn-Phe-Thr-Ile-Ser-Thr-pro-pro-pro-pro-Cys (19 aa). Represents a SARS2-CoV spike protein peptide mimitope partly homologous to NCBI-listed YP_009724390 residues 709-720.

Seq ID NO: 17. Cys-pro-pro-pro-pro-Ser-Ser-Asn-Leu-Leu-Leu-Gln-nPhe-Gly-Ser-Phe (16 aa). Represents a SARS2-CoV spike protein peptide mimitope partly homologous to NCBI-listed YP_009724390 residues 751-759.

Seq ID NO: 18. Gly-Gly-Phe-Asn-Phe-Ser-Gln-Ile-Leu-Pro-Asp-Pro-Ser-Ser-pro-pro-pro-pro-Cys (19 aa). Represents a SARS2-CoV spike protein peptide mimitope partly homologous to NCBI-listed YP_009724390 residues 798-809.

Seq ID NO: 19. Cys-pro-pro-pro-pro-Ser-Ser-Arg-Ser-Phe-Ile-Glu-Asp (13 aa). Represents a SARS2-CoV spike protein peptide mimitope partly homologous to NCBI-listed YP_009724390 residues 815-821.

Seq ID NO: 20. Cys-pro-pro-pro-pro-Thr-Thr-Ser-Thr-Ala-Leu-Gly-Lys-Leu-Gln-Asp (16 aa). Represents a SARS2-CoV spike protein peptide mimitope partly homologous to NCBI-listed YP_009724390 residues 942-950

Seq ID NO: 21. Gly-Val-Pro-Ile-Asn-Thr-Asn-Ser-Thr-Thr-pro-pro-pro-pro-Cys (15 aa). Represents a SARS2-CoV nucleocapsid protein peptide mimitope partly homologous to NCBI-listed QHD43423.2 residues 71-78.

Seq ID NO: 22. Cys-pro-pro-pro-pro-Ser-Ser-Pro-Arg-Trp-Tyr-Phe-Tyr-nPhe (14 aa). Represents a SARS2-CoV nucleocapsid protein peptide mimitope partly homologous to NCBI-listed QHD43423.2 residues 106-112.

Seq ID NO: 23. Gly-Thr-Arg-Asn-Pro-Ala-Asn-Asn-Thr-Thr-pro-pro-pro-pro-Cy (15 aa). Represents a SARS2-CoV nucleocapsid protein peptide mimitope partly homologous to NCBI-listed QHD43423.2 residues 147-154.

Seq ID NO: 24. Cys-pro-pro-pro-pro-Ser-Ser-Ser-Arg-Ser-Ser-Ser-Arg-Ser-Arg (15 aa). Represents a SARS2-CoV nucleocapsid protein peptide mimitope partly homologous to NCBI-listed QHD43423.2 residues 184-191.

Seq ID NO: 25. Ile-Asp-Ala-Tyr-Lys-Thr-Phe-Pro-Ser-Ser-pro-pro-pro-pro-Cys (15 aa). Represents a SARS2-CoV nucleocapsid protein peptide mimitope partly homologous to NCBI-listed QHD43423.2 residues 357-364.

Seq ID NO: 26. Ser-Lys-Arg-Ser-Phe-Val-Glu-Asp-Leu-Leu-Phe-Asn-Lys-Thr-Thr-pro-pro-pro-pro-Cys (20 aa). Represents a SARS2-CoV spike protein peptide mimitope partly homologous to NCBI-listed YP_009724390 residues 813-825.

Seq ID NO: 27. Thr-Leu-Ala-Asp-Val-Gly-Phe-nLeu-Lys-Gln-Tyr-Asp-Asp-Ser-Thr-pro-pro-pro-pro-Cys (20 aa). Represents a SARS2-CoV spike protein peptide mimitope partly homologous to NCBI-listed YP_009724390 residues 827-839.

Seq ID NO: 28. Cys-pro-pro-pro-pro-Ser-Thr-Ala-Gly-Ala-Ala-Ala-Tyr-Tyr-Val-Gly-nPhe (17 aa). Represents a SARS2-CoV spike protein peptide mimitope partly homologous to NCBI-listed YP_009724390 residues 260-269.

Seq ID NO: 29. Cys-pro-pro-pro-pro-Ser-Ser-Asp-Pro-Leu-Ser-Glu-Thr-Lys-Ser-Thr (16 aa). Represents a SARS2-CoV spike protein peptide mimitope partly homologous to NCBI-listed YP_009724390 residues 294-302.

Seq ID NO: 30. Gln-Lys-Leu-Ile-Ala-Asn-Gln-Phe-Asn-Ser-Ala-Ser-Ser-pro-pro-pro-pro-Cys (18 aa). Represents a SARS2-CoV spike protein peptide mimitope partly homologous to NCBI-listed YP_009724390 residues 920-930.

Seq ID NO: 31. His-Thr-Ser-Pro-Asp-Val-Asp-Leu-Gly-Asp-Ile-Ser-Gly-Thr-Thr-pro-pro-pro-pro-Cys (20 aa). Represents a SARS2-CoV spike protein peptide mimitope partly homologous to NCBI-listed YP_009724390 residues 1159-1171.

Seq ID NO: 32. Asn-Gly-Thr-Ile-Thr-Val-Glu-Glu-Leu-Lys-Lys-Leu-Leu-Glu-Gln-Trp-Asn-Thr-Thr-pro-pro-pro-pro-Cys (24 aa). Represents a SARS2-CoV Membrane protein peptide mimitope partly homologous to Uniprot-listed PODTC5 residues 5-21.

Seq ID NO: 33. Ser-Glu-Glu-Thr-Gly-Thr-Leu-Ile-Val-Asn-Ser-Thr-Thr-pro-pro-pro-pro-Cys (18aa). Represents a SARS2-CoV Envelope protein peptide mimitope partly homologous to Uniprot-listed residues PODTC4 residues 6-16.

Seq ID NO: 34. Cys-pro-pro-pro-pro-Thr-Thr-Pro-Asn-Asn-Thr-Ala-Ser-Trp-Phe-Thr-Ala-Leu-Thr-Gln-His-Gly-Lys (23 aa). Represents a SARS2-CoV nucleocapsid protein peptide mimitope partly homologous to NCBI-listed QHD43423.2 residues 46-61.

Seq ID NO: 35. Lys-His-Ile-Asp-Ala-Tyr-Lys-Thr-Phe-Pro-Pro-Thr-Thr-pro-pro-pro-pro-Cys (18 aa). Represents a SARS2-CoV nucleocapsid protein peptide mimitope partly homologous to NCBI-listed QHD43423.2 residues 355-365.

Seq ID NO: 36. Gln-Arg-Gln-Lys-Lys-Gln-Gln-Thr-Val-Thr-Leu-Leu-Pro-Ala-Ala-Ser-Thr-pro-pro-pro-pro-Cys (22 aa). Represents a SARS2-CoV nucleocapsid protein peptide mimitope partly homologous to NCBI-listed QHD43423.2 residues 384-398.

Seq ID NO: 37. Asn-Leu-Asn-Arg-Gly-nLeu-Val-Leu-Gly-Ser-Leu-Ala-Ser-Thr-pro-pro-pro-pro-Cys (19 aa). Represents a SARS2-CoV Replicase protein peptide mimitope partly homologous to Uniprot-listed residues PODTC1 residues 4236-4247.

Seq ID NO: 38. pro-pro-pro-pro-Cys.

Seq ID NO: 39. Cys-pro-pro-pro-pro.

In the above sequence listing, capital letters are L-isomers, and amino acid(s) in lower case are D-isomer amino acids); where M* is L-Norleucine (nLeu) the L-amino acid mimic for L-Methionine and where nPhe represents para-nitro-phenylalanine—a Y mimetic.

### REFERENCES

K. A. McLean et. al., “The 2015 Global Production Capacity of Seasonal and Pandemic Influenza Vaccine”. Vaccine 2016; 34 (45), 5410-5413.
S. Kalman et al., “COVID-19 (Novel Coronavirus 2019)—recent trends” Eur Rev Med Pharmacol Sci. 2020; 24(4):2006-2011.
D. Wrapp, et al. “Cryo-EM structure of the 2019-nCoV spike in the prefusion conformation”. Science. 2020; 367(6483):1260-1263.
Qing He et al., “Calcium Phosphate Nanoparticle Adjuvant” Clin. Diag. Lab Immunology 2000; 7:899-903.
FIELDS VIROLOGY, 6^{th} ed. Vol. I, D. M. Knipe & P. Howley (eds.), Lippincott Williams & Wilkins (2013).

1. An immunogenic composition comprising: (A) at least one mimetic peptide comprising: (i) at least one amino acid sequence conserved among beta coronaviruses, selected from the spike protein domain groups, nucleocapsid protein domains, Membrane matrix protein, Envelope protein, and/or Replicase protein, synthesized covalently to (ii) a 7 amino-acid spacer moiety, and (B) an immunogenic carrier coupled to said mimetic peptide, wherein the at least one mimetic peptide comprises a chemically synthesized and/or modified amino acid and/or amino acid epitope that augments the immunogenicity of the polypeptide immunogen, and wherein the amino acid sequence is from about 6 amino acid residues to about 30 amino acids, comprising a mixture of L- and D-enantiomeric amino acids.

2. The immunogenic composition according to claim 1, further comprising: at least 3-5 oligopeptides comprising amino acid sequences selected from the group consisting of SEQ ID NOs:1-20 and 26-31 and/or amino acid sequences and/or mimetic sequences selected from the group consisting of SEQ ID NOs: 21-25 and 34-36 and/or SEQ ID: 32, 33, and 37; and a pharmaceutically acceptable carrier.

3. The immunogenic composition according to claim 1, wherein the at least one mimetic peptide comprises: one or more oligopeptides having an overall length of from 6-50 amino acids, optionally from 6-30 amino acids, selected from the group consisting of SEQ ID:1-20 and 26-31 and amino acid sequences and/or mimetic sequences selected from the group consisting of SEQ ID: 21-25 and 34-36 and/or SEQ ID: 32, 33, and 37; and a pharmaceutically combination consisting of an acceptable carrier in a nano-sized emulsified preparation suitable for parenteral or mucosal application(s).

4. The immunogenic composition according to claim 1, wherein at least the immunogen(s) administered comprises a mixture of mimetic peptides selected from the group consisting of SEQ ID NOs:1-37.

5. The immunogenic composition according to claim 1, wherein the immunogenic carrier is selected from the group consisting of toxoidal proteins, diphtheria toxoid (DT), tetanus toxoid (TT), pertussin toxoid (PT), or recombinant toxoidal-like protein, a non-toxic but immunogenic recombinant Tetanus (toxoidal) protein, and nanoparticulate calcium phosphate (nCAP) carrier substance, with or without ancillary protein carriers.

6. The immunogenic composition according to claim 5, wherein the immunogenic carrier is tetanus toxoid.

7. An immunogenic composition comprising a therapeutically effective amount of the polypeptide immunogenic composition according to claim 1, and a pharmaceutically acceptable carrier.

8. The immunogenic composition according to claim 1, wherein said pharmaceutically acceptable carrier comprises an emulsion of an aqueous phase, in which said immunogen is dissolved or in suspension, and an oily phase.

9. The immunogenic composition according to claim 8, wherein said oily phase comprises at least one or more of squalene, squalane, sorbitan monooleate, Polysorbate 40, and Polysorbate 80.

10. The immunogenic composition according to claim 8, wherein said oily phase comprises at least one or more emulsifier agents.

11. The immunogenic composition according to claim 1, wherein either said oily phase or said aqueous phase contains at least one or more adjuvants.

12. The immunogenic composition according to claim 1, wherein said adjuvant is selected from the group consisting of cyclic-di-purine mononucleotides, cyclic diguanylate, Imiquimod, cyclic diadenylate, Isoprinosine, trehalose dimycolate, QS-21, alpha-galactosylceramide (C-GalCer), alpha-glucosylceramide (C-GluCer), and combinations thereof.

13. The immunogenic composition according to claim 12, wherein said adjuvant is selected from the group consisting of Ergamisol, Cimetidine, Praziquantel, uric acid, mannan and derivatives of mannan, and one or more of the vitamins A, D3 and vitamin E.

14. An immunization method comprising administering to patient having any beta-coronavirus infection an immunogenic composition according to claim 1, optionally wherein the immunogenic composition is administered in two doses.

15-29. (canceled)

30. A vaccine composition comprising at least one peptide comprising an amino acid sequence selected from the group consisting of SEQ ID NOs: 1-37, optionally coupled to a spacer peptide comprising two hydroxy amino acids coupled to either SEQ ID NO: 38 or 39.

31. The vaccine composition of claim 30, wherein the at least one peptide is further coupled to an immunogenic carrier.

32. The vaccine composition of claim 30, further comprising an adjuvant, excipient, and/or pharmaceutically acceptable carrier.

33. (canceled)

34. The vaccine composition of claim 30, wherein the vaccine composition comprises at least three peptides comprising an amino acid sequence selected from the group consisting of SEQ ID NOs: 1-37.

35-36. (canceled)

37. The vaccine composition of claim 30, wherein the vaccine composition comprises one or more of SEQ ID NOs: 10, 14, 15, 28, 30, and 31.

38-39. (canceled)


SEQUENCE LISTING (39 sequences)

SEQ ID NO: 1 - protein, 14 aa, Artificial Sequence
  Description of Artificial Sequence Synthetic peptide
  MOD_RES (10)..(10): D-amino acid
  MOD_RES (11)..(11): D-amino acid
  MOD_RES (12)..(12): D-amino acid
  MOD_RES (13)..(13): D-amino acid
RGVYYPDTTPPPPC

SEQ ID NO: 2 - protein, 13 aa, Artificial Sequence
  Description of Artificial Sequence Synthetic peptide
  MOD_RES (9)..(9): D-amino acid
  MOD_RES (10)..(10): D-amino acid
  MOD_RES (11)..(11): D-amino acid
  MOD_RES (12)..(12): D-amino acid
RGWIFGSTPPPPC

SEQ ID NO: 3 - protein, 15 aa, Artificial Sequence
  Description of Artificial Sequence Synthetic peptide
  MOD_RES (11)..(11): D-amino acid
  MOD_RES (12)..(12): D-amino acid
  MOD_RES (13)..(13): D-amino acid
  MOD_RES (14)..(14): D-amino acid
LREFVFKNSSPPPPC

SEQ ID NO: 4 - protein, 14 aa, Artificial Sequence
  Description of Artificial Sequence Synthetic peptide
  MOD_RES (10)..(10): D-amino acid
  MOD_RES (11)..(11): D-amino acid
  MOD_RES (12)..(12): D-amino acid
  MOD_RES (13)..(13): D-amino acid
RFPNITNSSPPPPC

SEQ ID NO: 5 - protein, 15 aa, Artificial Sequence
  Description of Artificial Sequence Synthetic peptide
  MOD_RES (11)..(11): D-amino acid
  MOD_RES (12)..(12): D-amino acid
  MOD_RES (13)..(13): D-amino acid
  MOD_RES (14)..(14): D-amino acid
PFGEVFNATTPPPPC

SEQ ID NO: 6 - protein, 15 aa, Artificial Sequence
  Description of Artificial Sequence Synthetic peptide
  MOD_RES (7)..(7): para-nitro-phenylalanine
  MOD_RES (11)..(11): D-amino acid
  MOD_RES (12)..(12): D-amino acid
  MOD_RES (13)..(13): D-amino acid
  MOD_RES (14)..(14): D-amino acid
VADYSVFNTSPPPPC

SEQ ID NO: 7 - protein, 13 aa, Artificial Sequence
  Description of Artificial Sequence Synthetic peptide
  MOD_RES (2)..(2): D-amino acid
  MOD_RES (3)..(3): D-amino acid
  MOD_RES (4)..(4): D-amino acid
  MOD_RES (5)..(5): D-amino acid
CPPPPTSSFSTFK

SEQ ID NO: 8 - protein, 15 aa, Artificial Sequence
  Description of Artificial Sequence Synthetic peptide
  MOD_RES (2)..(2): D-amino acid
  MOD_RES (3)..(3): D-amino acid
  MOD_RES (4)..(4): D-amino acid
  MOD_RES (5)..(5): D-amino acid
CPPPPTTNVYADSFV

SEQ ID NO: 9 - protein, 19 aa, Artificial Sequence
  Description of Artificial Sequence Synthetic peptide
  MOD_RES (2)..(2): D-amino acid
  MOD_RES (3)..(3): D-amino acid
  MOD_RES (4)..(4): D-amino acid
  MOD_RES (5)..(5): D-amino acid
CPPPPSSDEVRQIAPGQTG

SEQ ID NO: 10 - protein, 18 aa, Artificial Sequence
  Description of Artificial Sequence Synthetic peptide
  MOD_RES (4)..(4): para-nitro-phenylalanine
  MOD_RES (14)..(14): D-amino acid
  MOD_RES (15)..(15): D-amino acid
  MOD_RES (16)..(16): D-amino acid
  MOD_RES (17)..(17): D-amino acid
IADFNYKLPDDTTPPPPC

SEQ ID NO: 11 - protein, 15 aa, Artificial Sequence
  Description of Artificial Sequence Synthetic peptide
  MOD_RES (11)..(11): D-amino acid
  MOD_RES (12)..(12): D-amino acid
  MOD_RES (13)..(13): D-amino acid
  MOD_RES (14)..(14): D-amino acid
KPFERDISTTPPPPC

SEQ ID NO: 12 - protein, 20 aa, Artificial Sequence
  Description of Artificial Sequence Synthetic peptide
  MOD_RES (3)..(3): para-nitro-phenylalanine
  MOD_RES (16)..(16): D-amino acid
  MOD_RES (17)..(17): D-amino acid
  MOD_RES (18)..(18): D-amino acid
  MOD_RES (19)..(19): D-amino acid
QPFRVVVLSFELISSPPPPC

SEQ ID NO: 13 - protein, 18 aa, Artificial Sequence
  Description of Artificial Sequence Synthetic peptide
  MOD_RES (14)..(14): D-amino acid
  MOD_RES (15)..(15): D-amino acid
  MOD_RES (16)..(16): D-amino acid
  MOD_RES (17)..(17): D-amino acid
GGVSVITPGTNTSPPPPC

SEQ ID NO: 14 - protein, 19 aa, Artificial Sequence
  Description of Artificial Sequence Synthetic peptide
  MOD_RES (15)..(15): D-amino acid
  MOD_RES (16)..(16): D-amino acid
  MOD_RES (17)..(17): D-amino acid
  MOD_RES (18)..(18): D-amino acid
NIPIGAGISAYHTSPPPPC

SEQ ID NO: 15 - protein, 15 aa, Artificial Sequence
  Description of Artificial Sequence Synthetic peptide
  MOD_RES (4)..(4): L-Norleucine
  MOD_RES (11)..(11): D-amino acid
  MOD_RES (12)..(12): D-amino acid
  MOD_RES (13)..(13): D-amino acid
  MOD_RES (14)..(14): D-amino acid
AYTLSLGASSPPPPC

SEQ ID NO: 16 - protein, 19 aa, Artificial Sequence
  Description of Artificial Sequence Synthetic peptide
  MOD_RES (15)..(15): D-amino acid
  MOD_RES (16)..(16): D-amino acid
  MOD_RES (17)..(17): D-amino acid
  MOD_RES (18)..(18): D-amino acid
NNSIAIPTNFTISTPPPPC

SEQ ID NO: 17 - protein, 16 aa, Artificial Sequence
  Description of Artificial Sequence Synthetic peptide
  MOD_RES (2)..(2): D-amino acid
  MOD_RES (3)..(3): D-amino acid
  MOD_RES (4)..(4): D-amino acid
  MOD_RES (5)..(5): D-amino acid
  MOD_RES (13)..(13): para-nitro-phenylalanine
CPPPPSSNLLLQFGSF

SEQ ID NO: 18 - protein, 19 aa, Artificial Sequence
  Description of Artificial Sequence Synthetic peptide
  MOD_RES (15)..(15): D-amino acid
  MOD_RES (16)..(16): D-amino acid
  MOD_RES (17)..(17): D-amino acid
  MOD_RES (18)..(18): D-amino acid
GGFNFSQILPDPSSPPPPC

SEQ ID NO: 19 - protein, 13 aa, Artificial Sequence
  Description of Artificial Sequence Synthetic peptide
  MOD_RES (2)..(2): D-amino acid
  MOD_RES (3)..(3): D-amino acid
  MOD_RES (4)..(4): D-amino acid
  MOD_RES (5)..(5): D-amino acid
CPPPPSSRSFIED

SEQ ID NO: 20 - protein, 16 aa, Artificial Sequence
  Description of Artificial Sequence Synthetic peptide
  MOD_RES (2)..(2): D-amino acid
  MOD_RES (3)..(3): D-amino acid
  MOD_RES (4)..(4): D-amino acid
  MOD_RES (5)..(5): D-amino acid
CPPPPTTSTALGKLQD

SEQ ID NO: 21 - protein, 15 aa, Artificial Sequence
  Description of Artificial Sequence Synthetic peptide
  MOD_RES (11)..(11): D-amino acid
  MOD_RES (12)..(12): D-amino acid
  MOD_RES (13)..(13): D-amino acid
  MOD_RES (14)..(14): D-amino acid
GVPINTNSTTPPPPC

SEQ ID NO: 22 - protein, 14 aa, Artificial Sequence
  Description of Artificial Sequence Synthetic peptide
  MOD_RES (2)..(2): D-amino acid
  MOD_RES (3)..(3): D-amino acid
  MOD_RES (4)..(4): D-amino acid
  MOD_RES (5)..(5): D-amino acid
  MOD_RES (14)..(14): para-nitro-phenylalanine
CPPPPSSPRWYFYF

SEQ ID NO: 23 - protein, 15 aa, Artificial Sequence
  Description of Artificial Sequence Synthetic peptide
  MOD_RES (11)..(11): D-amino acid
  MOD_RES (12)..(12): D-amino acid
  MOD_RES (13)..(13): D-amino acid
  MOD_RES (14)..(14): D-amino acid
GTRNPANNTTPPPPC

SEQ ID NO: 24 - protein, 15 aa, Artificial Sequence
  Description of Artificial Sequence Synthetic peptide
  MOD_RES (2)..(2): D-amino acid
  MOD_RES (3)..(3): D-amino acid
  MOD_RES (4)..(4): D-amino acid
  MOD_RES (5)..(5): D-amino acid
CPPPPSSSRSSSRSR

SEQ ID NO: 25 - protein, 15 aa, Artificial Sequence
  Description of Artificial Sequence Synthetic peptide
  MOD_RES (11)..(11): D-amino acid
  MOD_RES (12)..(12): D-amino acid
  MOD_RES (13)..(13): D-amino acid
  MOD_RES (14)..(14): D-amino acid
IDAYKTFPSSPPPPC

SEQ ID NO: 26 - protein, 20 aa, Artificial Sequence
  Description of Artificial Sequence Synthetic peptide
  MOD_RES (16)..(16): D-amino acid
  MOD_RES (17)..(17): D-amino acid
  MOD_RES (18)..(18): D-amino acid
  MOD_RES (19)..(19): D-amino acid
SKRSFVEDLLFNKTTPPPPC

SEQ ID NO: 27 - protein, 20 aa, Artificial Sequence
  Description of Artificial Sequence Synthetic peptide
  MOD_RES (8)..(8): L-Norleucine
  MOD_RES (16)..(16): D-amino acid
  MOD_RES (17)..(17): D-amino acid
  MOD_RES (18)..(18): D-amino acid
  MOD_RES (19)..(19): D-amino acid
TLADVGFLKQYDDSTPPPPC

SEQ ID NO: 28 - protein, 17 aa, Artificial Sequence
  Description of Artificial Sequence Synthetic peptide
  MOD_RES (2)..(2): D-amino acid
  MOD_RES (3)..(3): D-amino acid
  MOD_RES (4)..(4): D-amino acid
  MOD_RES (5)..(5): D-amino acid
  MOD_RES (17)..(17): para-nitro-phenylalanine
CPPPPSTAGAAAYYVGF

SEQ ID NO: 29 - protein, 16 aa, Artificial Sequence
  Description of Artificial Sequence Synthetic peptide
  MOD_RES (2)..(2): D-amino acid
  MOD_RES (3)..(3): D-amino acid
  MOD_RES (4)..(4): D-amino acid
  MOD_RES (5)..(5): D-amino acid
CPPPPSSDPLSETKST

SEQ ID NO: 30 - protein, 18 aa, Artificial Sequence
  Description of Artificial Sequence Synthetic peptide
  MOD_RES (14)..(14): D-amino acid
  MOD_RES (15)..(15): D-amino acid
  MOD_RES (16)..(16): D-amino acid
  MOD_RES (17)..(17): D-amino acid
QKLIANQFNSASSPPPPC

SEQ ID NO: 31 - protein, 20 aa, Artificial Sequence
  Description of Artificial Sequence Synthetic peptide
  MOD_RES (16)..(16): D-amino acid
  MOD_RES (17)..(17): D-amino acid
  MOD_RES (18)..(18): D-amino acid
  MOD_RES (19)..(19): D-amino acid
HTSPDVDLGDISGTTPPPPC

SEQ ID NO: 32 - protein, 24 aa, Artificial Sequence
  Description of Artificial Sequence Synthetic peptide
  MOD_RES (20)..(20): D-amino acid
  MOD_RES (21)..(21): D-amino acid
  MOD_RES (22)..(22): D-amino acid
  MOD_RES (23)..(23): D-amino acid
NGTITVEELKKLLEQWNTTPPPPC

SEQ ID NO: 33 - protein, 18 aa, Artificial Sequence
  Description of Artificial Sequence Synthetic peptide
  MOD_RES (14)..(14): D-amino acid
  MOD_RES (15)..(15): D-amino acid
  MOD_RES (16)..(16): D-amino acid
  MOD_RES (17)..(17): D-amino acid
SEETGTLIVNSTTPPPPC

SEQ ID NO: 34 - protein, 23 aa, Artificial Sequence
  Description of Artificial Sequence Synthetic peptide
  MOD_RES (2)..(2): D-amino acid
  MOD_RES (3)..(3): D-amino acid
  MOD_RES (4)..(4): D-amino acid
  MOD_RES (5)..(5): D-amino acid
CPPPPTTPNNTASWFTALTQHGK

SEQ ID NO: 35 - protein, 18 aa, Artificial Sequence
  Description of Artificial Sequence Synthetic peptide
  MOD_RES (14)..(14): D-amino acid
  MOD_RES (15)..(15): D-amino acid
  MOD_RES (16)..(16): D-amino acid
  MOD_RES (17)..(17): D-amino acid
KHIDAYKTFPPTTPPPPC

SEQ ID NO: 36 - protein, 22 aa, Artificial Sequence
  Description of Artificial Sequence Synthetic peptide
  MOD_RES (18)..(18): D-amino acid
  MOD_RES (19)..(19): D-amino acid
  MOD_RES (20)..(20): D-amino acid
  MOD_RES (21)..(21): D-amino acid
QRQKKQQTVTLLPAASTPPPPC

SEQ ID NO: 37 - protein, 19 aa, Artificial Sequence
  Description of Artificial Sequence Synthetic peptide
  MOD_RES (6)..(6): L-Norleucine
  MOD_RES (15)..(15): D-amino acid
  MOD_RES (16)..(16): D-amino acid
  MOD_RES (17)..(17): D-amino acid
  MOD_RES (18)..(18): D-amino acid
NLNRGLVLGSLASTPPPPC

SEQ ID NO: 38 - protein, 5 aa, Artificial Sequence
  Description of Artificial Sequence Synthetic peptide
  MOD_RES (1)..(1): D-amino acid
  MOD_RES (2)..(2): D-amino acid
  MOD_RES (3)..(3): D-amino acid
  MOD_RES (4)..(4): D-amino acid
PPPPC

SEQ ID NO: 39 - protein, 5 aa, Artificial Sequence
  Description of Artificial Sequence Synthetic peptide
  MOD_RES (2)..(2): D-amino acid
  MOD_RES (3)..(3): D-amino acid
  MOD_RES (4)..(4): D-amino acid
  MOD_RES (5)..(5): D-amino acid
CPPPP